(19) United States
(12) Patent Application Publication
(10) Pub. No.: US 2025/0109503 A1
(43) Pub. Date: Apr. 3, 2025

(54) MALEATED FATTY IMIDAZOLINE DERIVATIVES FOR CORROSION INHIBITORS

(71) Applicant: ChampionX LLC, Sugar Land, TX (US)

(72) Inventors: Jing WU, Missouri City, TX (US); Shelley ZHOU, Sugar Land, TX (US); Jeremy MOLONEY, Katy, TX (US)

(73) Assignee: ChampionX LLC, Sugar Land, TX (US)

(21) Appl. No.: 18/829,923

(22) Filed: Sep. 10, 2024

Related U.S. Application Data
(60) Provisional application No. 63/586,827, filed on Sep. 29, 2023.

(51) Int. Cl. C23F 11/14; C07D 233/16

(52) U.S. Cl. CPC C23F 11/146; C07D 233/16

(57) ABSTRACT

Compositions and methods for inhibiting corrosion of metal surfaces are disclosed herein. Also disclosed are methods of manufacturing the corrosion inhibitors compositions. The corrosion inhibitor compositions include a reaction product of maleated fatty imidazoline formed from a fatty acid, e.g., maleated tall oil fatty acid and/or a maleated soya acid, with one or more amines. The reaction product can be further reacted with one or more of acrylic acid or acetic acid to form derivatives of a maleated fatty imidazoline acrylate compound or a maleated fatty imidazoline acetate compound, respectively.

### TECHNICAL FIELD

The present disclosure generally relates to methods and compositions for inhibiting corrosion of metallic surfaces.

### BACKGROUND

Aqueous liquids are injected into the earth and/or recovered from the earth during subterranean hydrocarbon recovery processes, such as hydraulic fracturing (fracking) and tertiary oil recovery. In some processes, an aqueous liquid called an “injectate” is injected into a subterranean formation and a water source called “produced water” is recovered, i.e., flows back from the subterranean formation and is collected along with a hydrocarbon product. The injectate and the produced water may include one or more corrodents, such as salts and/or other dissolved solids, liquids, or gases that cause, accelerate, or promote corrosion of metal surfaces and/or containments, such as metal pipelines and metal tanks.

Corrosion inhibitors are typically employed to reduce corrosion of metal surfaces that are contacted by liquids containing corrodents. For example, corrosion inhibitors can protect carbon steel pipelines and infrastructures from corrosion in oil and gas industries. Corrosion inhibitors are added to the liquids and dissolved gasses that come into contact with the metal surfaces and they act to prevent, retard, delay, reverse, and/or otherwise inhibit corrosion of the metal surfaces.

Sulfur-based compounds are known to be highly effective corrosion inhibitors and are favored because they are inexpensive. However, some sulfur-based corrosion inhibitors are known to produce hydrogen sulfide gas when stored in an enclosed space.

Hydrogen sulfide is a known corrodent recognized to cause severe corrosion issues. Hydrogen sulfide is toxic and dissolves in both hydrocarbon (oil/gasoline) and water streams. Further, hydrogen sulfide is a flammable gas, providing a severe health and safety risk.

Fatty imidazoline derivatized from tall oil fatty acid (TOFA) and maleated TOFA have conventionally been used as ingredients in corrosion inhibitors in the oil and gas industries for decades. However, using maleated TOFAs and/or fatty imidazolines can cause poor water partitioning, reducing performance at higher water cut and high temperature conditions.

### BRIEF SUMMARY

The present disclosure provides compositions and methods for inhibiting corrosion of metal surfaces. In some embodiments, a method of inhibiting corrosion of a metal surface in contact with a medium may include adding an effective amount of a composition to the medium. The composition may include a maleated fatty imidazoline, a maleated fatty imidazoline acrylate compound, a maleated fatty imidazoline acetate compound, or a combination thereof.

In some embodiments, the method comprises forming the maleated fatty imidazoline by reacting one or more of maleated tall oil fatty acid, soya acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, hexadecatrienoic acid, stearidonic acid, eicosenic acid, canola acid, eicosatrienoic acid, eicosatetraenoic acid, heneicosapentaenoic acid, clupanodonic acid, osbond acid, (9Z,12Z,15Z,18Z,21Z)-tetracosa-9,12,15,18,21-pentaenoic acid, nisinic acid, γ-linolenic acid, eicosadienoic acid, dihomo-γ-linolenic acid, docosadienoic acid, adrenic acid, tetracosatetraenoic acid, (6Z,9Z,12Z,15Z,18Z)-tetracosa-6,9,12,15,18-pentaenoic acid, (Z)-Eicos-11-enoic acid, mead acid, erucic acid, nervonic acid, rumenic acid, α-calendic acid, β-calendic acid, jacaric acid, α-eleostearic acid, β-eleostearic acid, catalpic acid, punicic acid, rumelenic acid, α-parinaric acid, β-parinaric acid, bosseopentaenoic acid, pinolenic acid, and/or podocarpic acid, or any combinations thereof, with one or more of diethylenetriamine (DETA), triethylenetetramine (TETA), and/or tetraethylenepentamine (TEPA) to form a reaction product. In some embodiments, forming the maleated fatty imidazoline may occur by reacting one or more of maleated tall oil fatty acid, soya acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, and hexatriacontylic acid. myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, hexadecatrienoic acid, stearidonic acid, eicosatrienoic acid, eicosatetraenoic acid, heneicosapentaenoic acid, clupanodonic acid, osbond acid, (9Z,12Z,15Z,18Z,21Z)-tetracosa-9,12,15,18,21-pentaenoic acid, nisinic acid, γ-linolenic acid, eicosadienoic acid, dihomo-γ-linolenic acid, docosadienoic acid, adrenic acid, tetracosatetraenoic acid, (6Z,9Z,12Z,15Z,18Z)-tetracosa-6,9,12,15,18-pentaenoic acid, (Z)-Eicos-11-enoic acid, mead acid, erucic acid, nervonic acid, rumenic acid, α-calendic acid, β-calendic acid, jacaric acid, α-eleostearic acid, β-eleostearic acid, catalpic acid, punicic acid, rumelenic acid, α-parinaric acid, β-parinaric acid, bosseopentaenoic acid, pinolenic acid, and/or podocarpic acid, or any combinations thereof, with a mixture of DETA, TETA, or TEPA to form a reaction product.

In some embodiments, the reaction product of the maleated tall oil fatty acid and the mixture of DETA, TETA, or TEPA can include the following

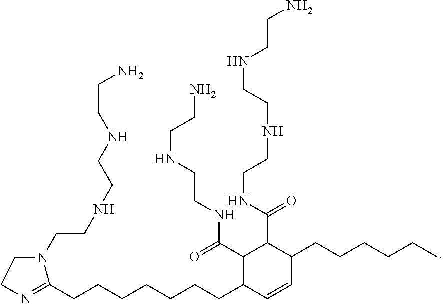 [SMILES: CCCCCCC1C=CC(CCCCCCCC2=NCCN2CCNCCNCCN)C(C(=O)NCCNCCN)C1C(=O)NCCNCCNCCN]

In some embodiments, the method can further include reacting the maleated soya acid with one or more of the DETA, TETA, and TEPA to form a reaction product.

In some embodiments, the reaction product of maleated soya acid and DETA can include the following structure:

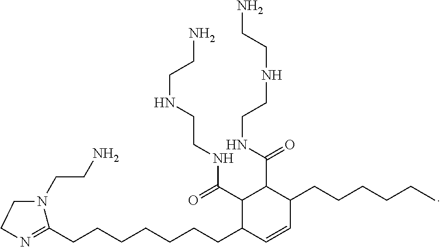 [SMILES: CCCCCCC1C=CC(CCCCCCCC2=NCCN2CCN)C(C(=O)NCCNCCN)C1C(=O)NCCNCCN]

In some embodiments, the reaction product of maleated soya acid and TETA can include the following structure:

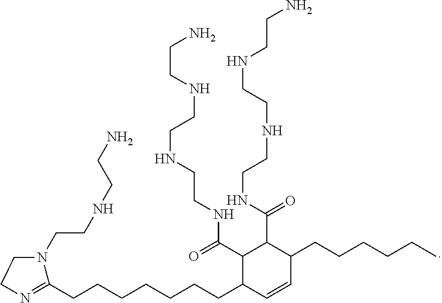 [SMILES: CCCCCCC1C=CC(CCCCCCCC2=NCCN2CCNCCN)C(C(=O)NCCNCCNCCN)C1C(=O)NCCNCCNCCN]

In some embodiments, the reaction product of maleated soya acid and TEPA can include the following structure:

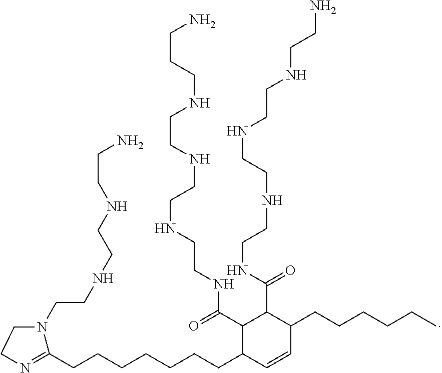 [SMILES: CCCCCCC1C=CC(CCCCCCCC2=NCCN2CCNCCNCCN)C(C(=O)NCCNCCNCCNCCCN)C1C(=O)NCCNCCNCCNCCN]

In some embodiments, the method may further include reacting the maleated soya acid with the mixture comprising DETA, TETA, and TEPA to form the reaction product.

Forming the maleated fatty imidazoline acrylate compound may occur by reacting the maleated tall oil fatty acid with one or more of DETA, TETA, and/or TEPA to form the reaction product and reacting the reaction product with acrylic acid. In some embodiments, the method may further comprise forming the maleated fatty imidazoline acrylate compound by reacting the maleated soya acid with one or more of DETA, TETA, and/or TEPA to form the reaction product and reacting the reaction product with acrylic acid. In some embodiments, forming the maleated fatty imidazoline acrylate compound may occur by reacting the maleated tall oil fatty acid with the mixture comprising DETA, TETA, and TEPA to form the reaction product and reacting the reaction product with acrylic acid. In some embodiments, the method can further include forming the maleated fatty imidazoline acrylate compound by reacting the maleated soya acid with the mixture of DETA, TETA, and TEPA to form the reaction product and reacting the reaction product with acrylic acid.

In some embodiments, the method can further include reacting the reaction product with the acrylic acid at a temperature between about 60° C. and about 120° C. Reacting the reaction product with the acrylic acid may occur at a molar ratio of about 0.3:1 to about 10:1.

In some embodiments, forming the maleated fatty imidazoline acetate compound may occur by reacting the maleated tall oil fatty acid with one or more of DETA, TETA, and/or TEPA to form the reaction product and reacting the reaction product with acetic acid.

In some embodiments, the method can further include forming the maleated fatty imidazoline acetate compound by reacting the maleated soya acid with one or more of DETA, TETA, and/or TEPA to form the reaction product and reacting the reaction product with acetic acid.

Forming the maleated fatty imidazoline acetate compound may occur by reacting the maleated tall oil fatty acid with the mixture comprising DETA, TETA, and TEPA to form the reaction product and reacting the reaction product with acetic acid.

In some embodiments, forming the maleated fatty imidazoline acetate compound may occur by reacting the maleated soya acid with the mixture of DETA, TETA, and TEPA to form the reaction product and reacting the reaction product with acetic acid.

In some embodiments, the method can further include reacting the reaction product with the acetic acid at a temperature in a range of about 20° C. to about 60° C. Reacting the reaction product with the acetic acid may occur at a molar ratio of about 0.3:1 to about 10:1.

The reaction product may comprise one or more of the following structures:

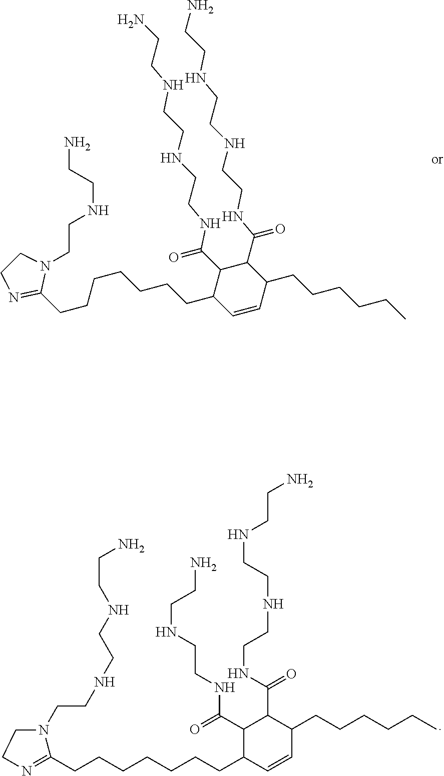 [SMILES: CCCCCCC1C=CC(CCCCCCCC2=NCCN2CCNCCN)C(C(=O)NCCNCCNCCN)C1C(=O)NCCNCCNCCN ; CCCCCCC1C=CC(CCCCCCCC2=NCCN2CCNCCNCCN)C(C(=O)NCCNCCN)C1C(=O)NCCNCCNCCN]

In some embodiments, the maleated fatty imidazoline acrylate compound may comprise the following structure:

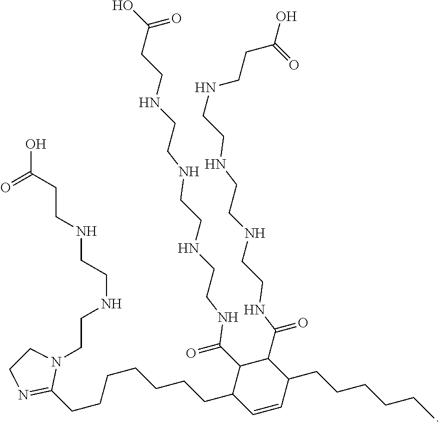 [SMILES: CCCCCCC1C=CC(CCCCCCCC2=NCCN2CCNCCNCCC(=O)O)C(C(=O)NCCNCCNCCNCCC(=O)O)C1C(=O)NCCNCCNCCNCCC(=O)O]

The method may further include adding 2-mercaptoethanol to the medium.

The effective amount of the compound may be from about 1 ppm to about 50,000 ppm.

In some embodiments, the medium may include a corrodent selected from the group consisting of hydrogen sulfide, carbon dioxide, oxygen, sodium chloride, calcium chloride, sulfur dioxide, elemental sulfur, organic acids, and any combination thereof. The medium may comprise, for example, produced water, fresh water, recycled water, salt water, surface water, condensed water, cooling water, injection water, wastewater, geothermal waters, sewage water, or any mixture thereof.

In certain embodiments, the medium may comprise from about 1 ppm to about 1,000 ppm of the compound.

The method may further include adding to the medium a component selected from the group consisting of a fouling control agent, an additional corrosion inhibitor, a biocide, a preservative, an acid, an anti-emulsifier, an iron chelating agent, a hydrogen sulfide scavenger, a surfactant, an asphaltene inhibitor, a paraffin inhibitor, a scale inhibitor, a gas hydrate inhibitor, a pH modifier, an emulsion breaker, a reverse emulsion breaker, a coagulant/flocculant agent, an emulsifier, a water clarifier, a dispersant, an antioxidant, a polymer degradation prevention agent, a permeability modifier, a foaming agent, an antifoaming agent, a CO₂ scavenger, an O₂ scavenger, a gelling agent, a lubricant, a friction reducing agent, a salt, and any combination thereof. The component may be added before, after, and/or with the composition. The composition may comprise from about 0.1 wt. % to about 20 wt. % of the component.

In some embodiments, the composition may comprise a solvent. The solvent may be selected from the group consisting of water, a C₁-C₆ alkanol, a C₁-C₆ alkoxyalkanol, an alcohol, a glycol ether, a hydrocarbon, a ketone, an ether, an aromatic, an alkylene glycol, an amide, a nitrile, a sulfoxide, an ester, and any combination thereof. The composition may comprise from about 1 wt. % to about 99 wt. % of the solvent.

In some embodiments, the composition may comprise from about 0.1 wt. % to about 100 wt. % of the maleated fatty imidazoline compound, the maleated fatty imidazoline acrylate compound, the maleated fatty imidazoline acetate compound, or the combination thereof. In some embodiments, the composition may comprise a pH from about 1 to about 11. In some embodiments, the metal surface may comprise carbon steel. One or more of a pipeline, a flowline, a downhole tubular, a casing, a tank, or a separator may comprise the metal surface.

The foregoing has outlined rather broadly the features and technical advantages of the present disclosure in order that the detailed description that follows may be better understood. Additional features and advantages of the disclosure will be described hereinafter that form the subject of the claims of this application. It should be appreciated by those skilled in the art that the conception and the specific embodiments disclosed may be readily utilized as a basis for modifying or designing other embodiments for carrying out the same purposes of the present disclosure. It should also be realized by those skilled in the art that such equivalent embodiments do not depart from the spirit and scope of the disclosure as set forth in the appended claims.

### DETAILED DESCRIPTION

The present disclosure provides compositions and methods for inhibiting corrosion of metallic surfaces. The disclosure also provides methods of manufacturing the corrosion inhibitor compositions.

Unless otherwise indicated, an alkyl group as described herein—alone or as part of another group—is an optionally substituted linear or branched saturated monovalent hydrocarbon substituent containing from, for example, one to about sixty carbon atoms, such as one to about thirty carbon atoms, in the main chain. Examples of unsubstituted alkyl groups include methyl, ethyl, n-propyl, i-propyl, n-butyl, i-butyl, s-butyl, t-butyl, n-pentyl, i-pentyl, s-pentyl, t-pentyl, and the like.

The terms “aryl” or “ar” as used herein alone or as part of another group (e.g., arylene) denote optionally substituted homocyclic aromatic groups, such as monocyclic or bicyclic groups containing from about 6 to about 12 carbons in the ring portion, such as phenyl, biphenyl, naphthyl, substituted phenyl, substituted biphenyl or substituted naphthyl. The term “aryl” also includes heteroaryl functional groups. It is understood that the term “aryl” applies to cyclic substituents that are planar and comprise 4n+2 electrons, according to Huckel's Rule.

“Cycloalkyl” refers to a cyclic alkyl substituent containing from, for example, about 3 to about 8 carbon atoms, preferably from about 4 to about 7 carbon atoms, and more preferably from about 4 to about 6 carbon atoms. Examples of such substituents include cyclopropyl, cyclobutyl, cyclopentyl, cyclohexyl, cycloheptyl, cyclooctyl, and the like. The cyclic alkyl groups may be unsubstituted or further substituted with alkyl groups, such as methyl groups, ethyl groups, and the like.

“Halogen” or “halo” refers to F, Cl, Br, and I.

“Heteroaryl” refers to a monocyclic or bicyclic 5- or 6-membered ring system, wherein the heteroaryl group is unsaturated and satisfies Huckel's rule. Non-limiting examples of heteroaryl groups include furanyl, thiophenyl, pyrrolyl, pyrazolyl, imidazolyl, 1,2,3-triazolyl, 1,2,4-triazolyl, isoxazolyl, oxazolyl, isothiazolyl, thiazolyl, 1,3,4-oxadiazol-2-yl, 1,2,4-oxadiazol-2-yl, 5-methyl-1,3,4-oxadiazole, 3-methyl-1,2,4-oxadiazole, pyridinyl, pyrimidinyl, pyrazinyl, triazinyl, benzofuranyl, benzothiophenyl, indolyl, quinolinyl, isoquinolinyl, benzimidazolyl, benzoxazolinyl, benzothiazolinyl, quinazolinyl, and the like.

Compounds of the present disclosure may be substituted with suitable substituents. The term “suitable substituent,” as used herein, is intended to mean a chemically acceptable functional group, preferably a moiety that does not negate the activity of the compounds. Such suitable substituents include, but are not limited to, halo groups, perfluoroalkyl groups, perfluoro-alkoxy groups, alkyl groups, alkenyl groups, alkynyl groups, hydroxy groups, oxo groups, mercapto groups, alkylthio groups, alkoxy groups, aryl or heteroaryl groups, aryloxy or heteroaryloxy groups, aralkyl or heteroaralkyl groups, aralkoxy or heteroaralkoxy groups, HO—(C—O)— groups, heterocylic groups, cycloalkyl groups, amino groups, alkyl- and dialkylamino groups, carbamoyl groups, alkylcarbonyl groups, alkoxycarbonyl groups, alkylaminocarbonyl groups, dialkylamino carbonyl groups, arylcarbonyl groups, aryloxy-carbonyl groups, alkylsulfonyl groups, and arylsulfonyl groups. In some embodiments, suitable substituents may include halogen, an unsubstituted C₁-C₁₂ alkyl group, an unsubstituted C₄-C₆ aryl group, or an unsubstituted C₁-C₁₀ alkoxy group. Those skilled in the art will appreciate that many substituents can be substituted by additional substituents.

The term “substituted” as in “substituted alkyl,” means that in the group in question (e.g., the alkyl group), at least one hydrogen atom bound to a carbon atom is replaced with one or more substituent groups, such as hydroxy (—OH), alkylthio, phosphino, amido (—CON(R_{A})(R_{B}), wherein R_{A} and R_{B} are independently hydrogen, alkyl, or aryl), amino (—N(R_{A})(R_{B}), wherein R_{A} and R_{B} are independently hydrogen, alkyl, or aryl), halo (fluoro, chloro, bromo, or iodo), silyl, nitro (—NO₂), an ether (—OR_{A} wherein R_{A} is alkyl or aryl), an ester (—OC(O)R_{A} wherein R_{A} is alkyl or aryl), keto (—C(O)R_{A} wherein R_{A} is alkyl or aryl), heterocyclo, and the like.

When the term “substituted” introduces a list of possible substituted groups, it is intended that the term apply to every member of that group. That is, the phrase “optionally substituted alkyl or aryl” is to be interpreted as “optionally substituted alkyl or optionally substituted aryl.”

The terms “polymer,” “copolymer,” “polymerize,” “copolymerize,” and the like include not only polymers comprising two monomer residues and polymerization of two different monomers together, but also include (co)polymers comprising more than two monomer residues and polymerizing together more than two or more other monomers. For example, a polymer as disclosed herein includes a terpolymer, a tetrapolymer, polymers comprising more than four different monomers, as well as polymers comprising, consisting of, or consisting essentially of two different monomer residues. Additionally, a “polymer” as disclosed herein may also include a homopolymer, which is a polymer comprising a single type of monomer unit.

Unless specified differently, the polymers of the present disclosure may be linear, branched, crosslinked, structured, synthetic, semi-synthetic, natural, and/or functionally modified. A polymer of the present disclosure can be in the form of a solution, a dry powder, a liquid, or a dispersion, for example.

The present disclosure provides compositions and methods that can be used in industrial aqueous systems. In some embodiments, the compositions and methods may be used to inhibit corrosion of a metallic surface present in an oil and gas production well, a storage tank, and/or a pipeline, for example.

In some embodiments, the technology disclosed herein effectively enhances corrosion inhibitor performance and leads to film persistency/increased lifetime of the applied film. The technology, therefore, may decrease the frequency of needed batch corrosion inhibitor applications, which reduces cost and chemical usage.

The corrosion inhibitor compositions disclosed herein may comprise a variety of treatment chemicals and/or compounds, such as a corrosion inhibitor compound. Examples of corrosion inhibitor compounds include, but are not limited to, an organic sulfur compound, an imidazoline, a carboxylic acid-containing compound, a fatty acid amine condensate, a substituted fatty acid ester, a substituted aromatic amine, a phosphoric acid ester, a quaternary ammonium compound, or a compound comprising multiple positive charges.

In some embodiments, a corrosion inhibitor compound may be selected from a maleated fatty imidazoline compound, a maleated fatty imidazoline acrylate compound, a maleated fatty imidazoline acetate compound, or any combination thereof.

These compounds can enhance corrosion performance as compared to conventional corrosion inhibitors and improve the corrosion rate. Specifically, maleated fatty imidazoline and its maleated fatty imidazoline acrylate and/or maleated fatty imidazoline acetate derivatives are examples of compounds that can provide superior corrosion performance, particularly under broad flow, e.g., high/low shear, conditions. Several formulations of maleated fatty imidazoline acrylate and/or maleated fatty imidazoline acetate are discussed in greater detail below. These compounds offer a number of benefits, such as being environmentally friendly, high water-partitioning, high temperature stability, and/or cost effective.

At least one novel feature of the present embodiments includes adding an effective amount of a composition that includes the maleated fatty imidazoline compound, the maleated fatty imidazoline acrylate compound, the maleated fatty imidazoline acetate compound, or a combination thereof to a medium for purposes of inhibiting corrosion of a metal surface in contact therewith. As an illustrative, non-limiting example, the present disclosure provides Composition 1, which is a maleated fatty imidazoline reaction product of maleated TOFA and/or maleated soya acids with one or more of DETA, TETA, and TEPA in various combinations. That is, in some embodiments, the maleated fatty imidazoline of the present disclosure can be a reaction product of maleated TOFA with one or more of DETA, TETA, and TEPA, a reaction product of maleated TOFA with a mixture of two or three of DETA, TETA, and TEPA, a reaction product of maleated soya acids with one or more of DETA, TETA, and TEPA, and/or a reaction product of maleated soya acids with a mixture of DETA, TETA, and TEPA, and the like.

In some embodiments, additional fatty acids can be used in lieu of, and/or in addition to, one of maleated TOFA and/or maleated soya acids. For example, one or more of butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, hexadecatrienoic acid, stearidonic acid, eicosenic acid, canola acid, eicosatrienoic acid, eicosatetraenoic acid, heneicosapentaenoic acid, clupanodonic acid, osbond acid, (9Z,12Z,15Z,18Z,21Z)-tetracosa-9,12,15,18,21-pentaenoic acid, nisinic acid, γ-linolenic acid, eicosadienoic acid, dihomo-γ-linolenic acid, docosadienoic acid, adrenic acid, tetracosatetraenoic acid, (6Z,9Z,12Z,15Z,18Z)-tetracosa-6,9,12,15,18-pentaenoic acid, (Z)-Eicos-11-enoic acid, mead acid, erucic acid, nervonic acid, rumenic acid, α-calendic acid, β-calendic acid, jacaric acid, α-eleostearic acid, β-eleostearic acid, catalpic acid, punicic acid, rumelenic acid, α-parinaric acid, β-parinaric acid, bosseopentaenoic acid, pinolenic acid, and/or podocarpic acid, any combinations thereof, and/or any other suitable fatty acids, can be used in the reaction with one or more of DETA, TETA, and/or TEPA, or any mixture of DETA, TETA, and TEPA, to form the reaction product of the present disclosure.

One example of Composition 1, which can be referred to as Composition 1A, and can form as a reaction product of maleated TOFA and/or maleated soya acid with one or more of DETA, TETA, and/or TEPA, can have the following structure:

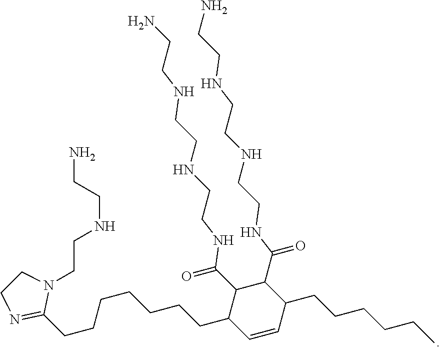 [SMILES: CCCCCCC1C=CC(CCCCCCCC2=NCCN2CCNCCN)C(C(=O)NCCNCCNCCN)C1C(=O)NCCNCCNCCN]

An additional non-limiting example of Composition 1, which may be referred to as Composition 1B (CAS No. 68990-47-6: fatty acids, tall-oil reaction products with diethylenetriamine, maleic anhydride, triethylenetetramine and tetraethylenepentamine), may have the following structure:

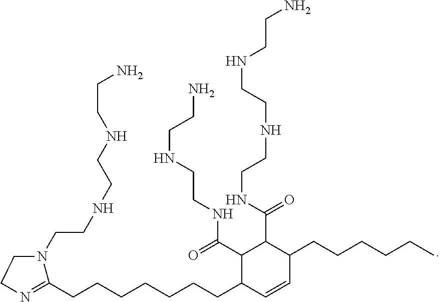 [SMILES: CCCCCCC1C=CC(CCCCCCCC2=NCCN2CCNCCNCCN)C(C(=O)NCCNCCN)C1C(=O)NCCNCCNCCN]

A person skilled in the art will recognize that the above structures of the maleated fatty imidazoline reaction product are merely examples and can vary based on the type of reagents used. For example, the maleated fatty imidazoline reaction product can result from a reaction of a maleated soya acid with a mixture of DETA, TETA, and TEPA, without a maleated TOFA, or a reaction of maleated soya acid with one or two of DETA, TETA, and TEPA, which would alter the structure of the maleated fatty imidazoline reaction product. As a non-limiting example, a reaction product resulting from a reaction of maleated soya acid with DETA can be a soya fatty acid with diethylenetriamine and maleic anhydride having the following structure:

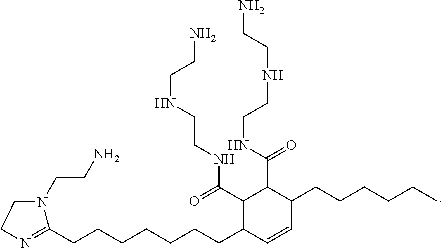 [SMILES: CCCCCCC1C=CC(CCCCCCCC2=NCCN2CCN)C(C(=O)NCCNCCN)C1C(=O)NCCNCCN]

In some embodiments, a reaction product resulting from a reaction of maleated soya acid with TETA can be soya fatty acid with triethylenetetramine and maleic anhydride having the following structure:

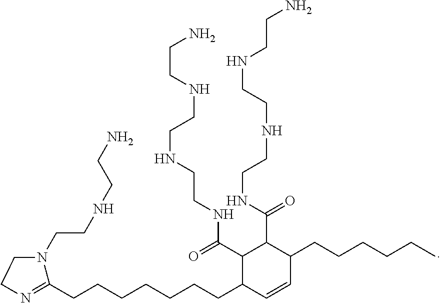 [SMILES: CCCCCCC1C=CC(CCCCCCCC2=NCCN2CCNCCN)C(C(=O)NCCNCCNCCN)C1C(=O)NCCNCCNCCN]

Moreover, in some embodiments, a reaction product resulting from a reaction of maleated soya acid with TEPA can be a soya fatty acid with tetraethylenepentamine and maleic anhydride having the following structure:

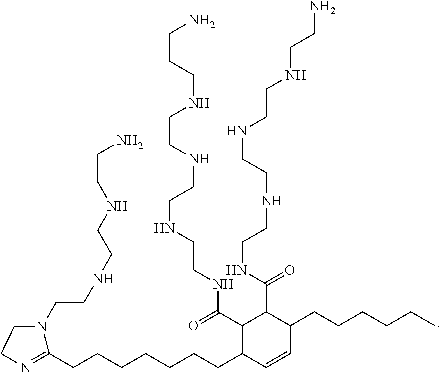 [SMILES: CCCCCCC1C=CC(CCCCCCCC2=NCCN2CCNCCNCCN)C(C(=O)NCCNCCNCCNCCCN)C1C(=O)NCCNCCNCCNCCN]

The structure of these maleated fatty imidazoline reaction products, as well as the structure of Composition 1, formed as a result of the reaction of the various reagent combinations discussed herein, can exhibit excellent performance as a corrosion inhibitor and are within the scope of the novel and inventive compounds and compositions of the present disclosure.

While Composition 1 may be discussed with respect to its involvement in further reactions, a person skilled in the art will appreciate that any of the maleated fatty imidazoline reaction products of the present disclosure can be involved in the reaction to prepare derivatives therefrom.

Composition 1 can react with various other compounds to form maleated fatty imidazoline derivatives. For example, in some embodiments, Composition 1 can react with acrylic acid to form Composition 2, which is a maleated fatty imidazoline acrylate having the following structure:

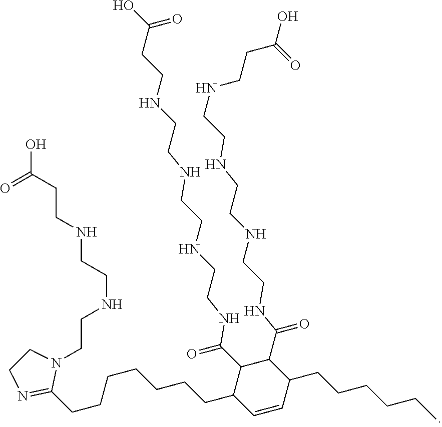 [SMILES: CCCCCCC1C=CC(CCCCCCCC2=NCCN2CCNCCNCCC(=O)O)C(C(=O)NCCNCCNCCNCCC(=O)O)C1C(=O)NCCNCCNCCNCCC(=O)O]

The reaction of Composition 1 with acrylic acid can occur in a range of about 60° C. to about 120° C., and/or at a temperature of about 100° C. The reaction can occur under a molar ratio of Composition 1: acrylic acid that is in a range of about 0.3:1 to about 10:1, such as from about 0.5:1 to about 5:1, about 0.7:1 to about 3:1, or about 1:1.

A person skilled in the art will recognize that the above structure of the maleated fatty imidazoline acrylate is merely an example of an embodiment and can vary based on the type of reagents used. For example, as discussed herein, the maleated fatty imidazoline acrylate can result from a reaction of Composition 1 with acrylic acid and/or any maleated fatty imidazoline discussed above with acrylic acid. The structure of these maleated fatty imidazoline acrylate compounds formed as a result of the reaction of the various reagent combinations discussed herein can exhibit excellent performance as corrosion inhibitors and are within the scope of the novel and inventive compounds and compositions of the present disclosure.

Another example of a maleated fatty imidazoline derivative can be formed when Composition 1 reacts with acetic acid to form Composition 3, which is a maleated fatty imidazoline acetate having the following formula:

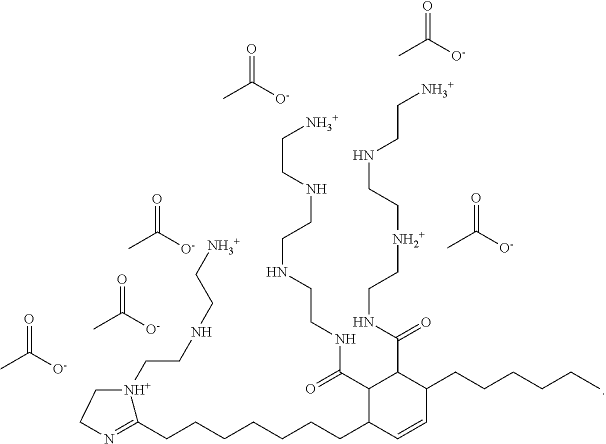

It will be appreciated that in some embodiments, glacial acetic acid can be used, though this reaction can occur with any form of acetic acid. The reaction of Composition 1 with acetic acid can occur at about room temperature (about 20° C. to about 22° C.), for example. The reaction can occur with a molar ratio of Composition 1: acetic acid of about 0.3:1 to about 10:1, such as from about 0.5:1 to about 7:1, about 0.7:1 to about 5:1, or about 1:1 to about 3:1.

A person skilled in the art will recognize that the above structure of the maleated fatty imidazoline acetate is merely an example and can vary based on the type of reagents used. For example, as discussed above, the maleated fatty imidazoline acetate can result from a reaction of Composition 1 with acetic acid and/or any maleated fatty imidazoline discussed above with acetic acid. The structure of these maleated fatty imidazoline acetate compounds formed as a result of the reaction of the various reagent combinations discussed above can exhibit excellent performance as corrosion inhibitors and are within the scope of the novel and inventive compounds of the present disclosure.

In some embodiments, a composition disclosed herein comprises, consists of, or consists essentially of, a maleated fatty imidazoline compound, a maleated fatty imidazoline acrylate compound, a maleated fatty imidazoline acetate compound, or any combination thereof, and optionally a quaternary amine, an iron chelating agent, and/or a solvent.

The imidazoline compound may have formula (I), (II), or (III):

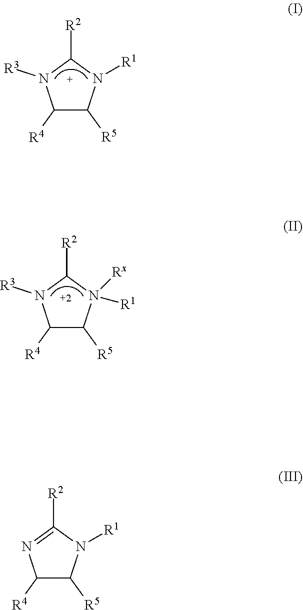

wherein R¹, R⁴, and R⁵ are each independently selected from hydrogen, alkyl, alkenyl, alkynyl, cycloalkyl, cycloalkenyl, aryl, heteroaryl, and heterocycle, said alkyl, alkenyl, alkynyl, cycloalkyl, cycloalkenyl, aryl, heteroaryl, and heterocycle each independently, at each occurrence, unsubstituted or substituted with 1 to 3 substituents independently selected from halogen, —COR⁶, —CO₂R⁷, —SO₃R⁸, —PO₃H₂, —CON(R⁹)(R¹⁰), —OR¹¹, and —N(R¹²)(R¹³);
R² is a radical derived from a fatty acid;
R³ and Rx are each independently selected from a radical derived from an unsaturated acid;
R⁶, R⁷, R⁸, R⁹, R¹⁰, and R¹¹ are each independently, at each occurrence, selected from hydrogen, alkyl, and alkenyl;
R¹² and R₁₃ are each independently, at each occurrence, selected from hydrogen, alkyl, —COR¹⁴, —CO₂R¹⁵, -alkyl-COR¹⁶, and -alkyl-CO₂R¹⁷; and
R¹⁴, R¹⁵, R¹⁶, and R¹⁷ are each independently, at each occurrence, selected from hydrogen, alkyl, and alkenyl.

In the foregoing imidazolines, R groups of carboxylic acid moieties can be absent where the R═H and the carboxylic acid moiety is deprotonated. For example, R¹⁵ and/or R¹⁷ can be absent where the R¹² and/or R¹³ is a deprotonated carboxylic acid moiety (e.g., where R¹² is —CH₂CH₂CO₂⁻). For an imidazoline compound, R¹ can be unsubstituted alkyl. For example, R¹ can be unsubstituted C₁-C₁₀-alkyl (e.g., methyl, ethyl, propyl (e.g., n-propyl, isopropyl), butyl (e.g., n-butyl, isobutyl, tert-butyl, sec-butyl), pentyl (e.g., n-pentyl, isopentyl, tert-pentyl, neopentyl, sec-pentyl, 3-pentyl), hexyl, heptyl, octyl, nonyl, or decyl). Further, R¹ can be unsubstituted C₂-C₁₀-alkyl. For the imidazoline compounds, R¹ can be unsubstituted C₂-C₈-alkyl. Further, R¹ can be unsubstituted C₂-C₆-alkyl. In some embodiments, R¹ is propyl, butyl, or hexyl.

In some embodiments, R¹ is a substituted alkyl. For example, R¹ may be a substituted C₁-C₁₀-alkyl, substituted C₂-C₁₀-alkyl, substituted C₂-C₈-alkyl, or substituted C₂-C₆-alkyl. Further, R¹ may be a C₁-C₁₀-alkyl, C₂-C₁₀-alkyl, C₂-C₈-alkyl, or C₂-C₆-alkyl, substituted with one substituent selected from —COR⁶, —CO₂R⁷, —SO₃R⁸, —PO₃H₂, —CON(R⁹)(R¹⁰), —OR¹¹, and —N(R¹²)(R¹³), wherein R⁶, R⁷, R⁸, R⁹, R¹⁰, R¹¹, R¹², and R¹³ are as defined above. More specifically, R¹ may be a C₂-C₆-alkyl, substituted with one substituent selected from —N(R¹²)(R¹³), wherein R¹² and R₁₃ are each independently selected from hydrogen, alkyl, —COR¹⁴, —CO₂R¹⁵, -alkyl-COR¹⁶, and -alkyl-CO₂R¹⁷, wherein R¹⁴, R¹⁵, R¹⁶, and R¹⁷ are as defined above. Further, R¹ may be a C₂-C₆-alkyl, substituted with one substituent selected from —N(R¹²)(R¹³), wherein R¹² and R₁₃ are each independently selected from hydrogen, C₂-C₆-alkyl, —COR¹⁴, —CO₂R¹⁵, —C₂-C₆-alkyl-COR¹⁶, and —C₂-C₆-alkyl-CO₂R¹⁷, wherein R¹⁴, R¹⁵, R¹⁶, and R¹⁷ are selected from hydrogen and C₁-C₃₄-alkyl. For these imidazolines, R¹ may be a linear C₂-C₆-alkyl, substituted with one substituent that is a terminal-N(R¹²)(R¹³), wherein R¹² and R₁₃ are each independently selected from hydrogen, —COR¹⁴, —CO₂R¹⁵, —C₂-C₆-alkyl-COR¹⁶, and —C₂-C₆-alkyl-CO₂R¹⁷, wherein R¹⁴, R¹⁵, R¹⁶, and R¹⁷ are selected from hydrogen and C₁-C₃₄-alkyl. For example, R¹ may be a linear C₂-alkyl, substituted with one substituent that is a terminal-N(R¹²)(R¹³), wherein R¹² is hydrogen and R¹³ is —COR¹⁴, wherein R¹⁴ is —C₁₇H₃₅, —C₁₇H₃₃, or —C₁₇H₃₁. Further, R¹ may be a linear C₂-alkyl, substituted with one substituent that is a terminal —N(R¹²)(R¹³), wherein R¹² and R₁₃ are each α-C₂-alkyl-CO₂R¹⁷, wherein R¹⁷ is hydrogen.

For the imidazolines of formulae (I), (II), and (III), R² may be a C₄-C₃₄-alkyl or C₄-C₃₄-alkenyl. For example, R² may be a —(CH₂)₃CH₃; —(CH₂)₄CH₃; —(CH₂)₅CH₃; —(CH₂)₆CH₃; —(CH₂)₇CH₃; —(CH₂)₈CH₃; —(CH₂)₉CH₃; —(CH₂)₁₀CH₃; —(CH₂)₁₁CH₃; —(CH₂)₁₂CH₃; —(CH₂)₁₃CH₃; —(CH₂)₁₄CH₃; —(CH₂)₁₅CH₃; —(CH₂)₁₆CH₃; —(CH₂)₁₇CH₃; —(CH₂)₁₈CH₃; —(CH₂)₁₉CH₃; —(CH₂)₂₀CH₃; —(CH₂)₂₁CH₃; —(CH₂)₂₂CH₃; —(CH₂)₂₃CH₃; —(CH₂)₂₄CH₃; —(CH₂)₂₅CH₃; —(CH₂)₂₆CH₃; —(CH₂)₂₇CH₃; —(CH₂)₂₈CH₃; —(CH₂)₂₉CH₃; —(CH₂)₃₀CH₃; —(CH₂)₃₁CH₃; —(CH₂)₃₂CH₃; —(CH₂)₃₃CH₃; —(CH₂)₃₄CH₃; —(CH₂)₂CH═CHCH₂CH═CHCH₂CH═CHCH₂CH═CHCH₂CH═CH(CH₂)₄CH₃; —(CH₂)₂CH═CHCH₂CH═CHCH₂CH—CHCH₂CH═CHCH₂CH═CHCH₂CH═CHCH₂CH₃; —(CH₂)₃CH═CHCH₂CH═CHCH₂CH═CH(CH₂)₇CH₃; —(CH₂)₃CH═CHCH₂CH₂CH═CHCH₂CH═CH(CH₂)₄CH₃; —(CH₂)₃CH═CH(CH₂)₄CH═CHCH₂CH═CH(CH₂)₄CH₃; —(CH₂)₃CH═CHCH₂CH═CHCH₂CH—CHCH₂CH═CH(CH₂)₄CH₃; —(CH₂)₃CH═CHCH₂CH═CHCH₂CH—CHCH₂CH—CHCH₂CH═CHCH₂CH₃; —(CH₂)₃CH═CHCH═CHCH—CHCH—CHCH═CH(CH₂)₄CH₃; —(CH₂)₄CH═CH(CH₂)₈CH₃; —(CH₂)₄CH═CHCH₂CH═CHCH₂CH═CH(CH₂)₄CH₃; —(CH₂)₄CH═CHCH₂CH═CHCH₂CH═CHCH₂CH═CHCH₂CH₃; —(CH₂)₄CH═CHCH₂CH═CHCH₂CH═CHCH₂CH═CHCH₂CH═CHCH₂CH₃; —(CH₂)₄CH═CHCH₂CH═CHCH₂CH═CHCH₂CH═CHCH₂CH═CH(CH₂)₄CH₃; —(CH₂)₄CH═CHCH₂CH—CHCH₂CH═CHCH₂CH═CHCH₂CH═CHCH₂CH═CHCH₂CH₃; —(CH₂)₅CH—CHCH₂CH—CHCH₂CH═CHCH₂CH₃; —(CH₂)₅CH═CHCH₂CH═CHCH₂CH═CHCH₂CH═CH(CH₂)₄CH₃; —(CH₂)₅CH—CHCH₂CH═CHCH₂CH═CHCH₂CH═CHCH₂CH═CHCH₂CH₃; —(CH₂)₆CH═CHCH═CHCH═CH(CH₂)₄CH₃; —(CH₂)₆CH═CHCH₂CH—CHCH₂CH—CHCH₂CH═CH(CH₂)₄CH₃; —(CH₂)₇CH═CH(CH₂)₃CH₃; —(CH₂)₇CH═CH(CH₂)₅CH₃; —(CH₂)₇CH═CH(CH₂)₇CH₃; —(CH₂)₇CH═CHCH═CHCH═CH(CH₂)₃CH₃; —(CH₂)₇CH═CHCH═CH(CH₂)₅CH₃; —(CH₂)₇CH═CHCH₂CH═CH(CH₂)₄CH₃; —(CH₂)₇CH═CHCH₂CH═CH(CH₂)₄CH₃; —(CH₂)₇CH═CHCH═CHCH₂CH₂CH═CHCH₂CH₃; —(CH₂)₇CH═CHCH═CHCH═CHCH—CHCH₂CH₃; —(CH₂)₇CH═CHCH₂CH═CHCH₂CH═CHCH₂CH═CH(CH₂)₄CH₃; —(CH₂)₇CH═CHCH₂CH═CHCH₂CH═CHCH₂CH₃; —(CH₂)₇CH═CHCH₂CH═CHCH₂CH═CHCH₂CH═CHCH₂CH═CHCH₂CH₃; —(CH₂)₉CH═CH(CH₂)₅CH₃; —(CH₂)₉CH═CHCH₂CH═CH(CH₂)₄CH₃; —(CH₂)₉CH═CHCH₂CH═CHCH₂CH═CHCH₂CH₃; —(CH₂)₉CH═CH(CH₂)₇CH₃; —(CH₂)₁₁CH═CH(CH₂)₅CH₃; —(CH₂)₁₁CH═CH(CH₂)₇CH₃; —(CH₂)₁₁CH═CHCH₂CH═CH(CH₂)₄CH₃; or —(CH₂)₁₃CH═CH(CH₂)₇CH₃.

In some embodiments, R² may be a radical derived from a saturated or unsaturated fatty acid. Suitable saturated fatty acids include, but are not limited to, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, and hexatriacontylic acid. Suitable unsaturated fatty acids include, but are not limited to, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, hexadecatrienoic acid, stearidonic acid, eicosenic acid, canola acid, eicosatrienoic acid, eicosatetraenoic acid, heneicosapentaenoic acid, clupanodonic acid, osbond acid, (9Z,12Z,15Z,18Z,21Z)-tetracosa-9,12,15,18,21-pentaenoic acid, nisinic acid, γ-linolenic acid, eicosadienoic acid, dihomo-γ-linolenic acid, docosadienoic acid, adrenic acid, tetracosatetraenoic acid, (6Z,9Z,12Z,15Z,18Z)-tetracosa-6,9,12,15,18-pentaenoic acid, (Z)-Eicos-11-enoic acid, mead acid, erucic acid, nervonic acid, rumenic acid, α-calendic acid, β-calendic acid, jacaric acid, α-eleostearic acid, β-eleostearic acid, catalpic acid, punicic acid, rumelenic acid, α-parinaric acid, β-parinaric acid, bosseopentaenoic acid, pinolenic acid, and podocarpic acid. In some embodiments, R² is derived from coconut oil, beef tallow, or tall oil fatty acids (TOFA).

In some embodiments, R³ may be —C(R^{a}R^{b})—C(R^{c}R^{d})—CO₂R^{e}, wherein R^{a}, R^{b}, R^{c}, and R^{d} are each independently selected from the group consisting of hydrogen (—H), halogen, and alkyl, and wherein R^{e} is hydrogen (—H) or alkyl. For example, R³ may be —C(R^{a}R^{b})—C(R^{c}R^{d})—CO₂R^{e}, wherein R^{a}, R^{b}, R^{c}, and R^{d} are each independently selected from the group consisting of hydrogen (—H), halogen, and C₁-C₆-alkyl, and wherein R^{e} is hydrogen (—H) or C₁-C₆-alkyl. Further, R³ may be —CH₂CH₂CO₂R^{e}, wherein R^{e} is hydrogen (—H) or C₁-C₆-alkyl. Additionally, R^{e} can be absent where the R³ is a deprotonated carboxylic acid moiety (e.g., where R³ is —CH₂CH₂CO₂⁻).

In accordance with certain embodiments of the present disclosure, R³ can be derived from an acrylic acid. Suitable acrylic acids include, but are not limited to, acrylic acid, methacrylic acid, 2-ethylacrylic acid, 2-propylacrylic acid, and 2-(trifluoromethyl) acrylic acid. For example, R³ can be derived from acrylic acid (H₂C═CHCO₂H).

Imidazolines of formulae (I), (II), or (III) may have R^{x} equal to —C(R^{a}R^{b})—C(R^{c}R^{d})—CO₂R^{e}, wherein R^{a}, R^{b}, R^{c}, and R^{d} are each independently selected from the group consisting of hydrogen (—H), halogen, and alkyl, and wherein R^{e} is hydrogen (—H) or alkyl. Further, R^{x} can be —C(R^{a}R^{b})—C(R^{c}R^{d})—CO₂R^{e}, wherein R^{a}, R^{b}, R^{c}, and R^{d} are each independently selected from the group consisting of hydrogen (—H), halogen, and C₁-C₆-alkyl, and wherein R^{e} is hydrogen (—H) or C₁-C₆-alkyl. Additionally, R^{x} may be —CH₂CH₂CO₂R^{e}, wherein R^{e} is hydrogen (—H) or C₁-C₆-alkyl. Further, R^{e} can be absent where the R^{x} is a deprotonated carboxylic acid moiety (e.g., where R^{x} is —CH₂CH₂CO₂⁻).

For the imidazolines described herein, R^{x} can be derived from an acrylic acid. Suitable acrylic acids include, but are not limited to, acrylic acid, methacrylic acid, 2-ethylacrylic acid, 2-propylacrylic acid, and 2-(trifluoromethyl) acrylic acid. For example, R^{x} can be derived from acrylic acid (H₂C═CHCO₂H).

Imidazolines of formulae (I), (II), or (III) can have R⁴ and R⁵ each independently be an unsubstituted C₁-C₁₀-alkyl (e.g., methyl, ethyl, propyl (e.g., n-propyl, isopropyl), butyl (e.g., n-butyl, isobutyl, tert-butyl, sec-butyl), pentyl (e.g., n-pentyl, isopentyl, tert-pentyl, neopentyl, sec-pentyl, 3-pentyl), hexyl, heptyl, octyl, nonyl, or decyl) or hydrogen. Further, R⁴ and R⁵ can each independently be an unsubstituted C₁-C₆ alkyl group or hydrogen. In some embodiments, R⁴ and R⁵ are each hydrogen (—H).

Imidazolines of formulae (I), (II), or (III) can have R⁶, R⁷, R⁸, R⁹, R¹⁰, and R¹¹ each independently be, at each occurrence, selected from hydrogen, unsubstituted alkyl, and unsubstituted alkenyl. For example, R⁶, R⁷, R⁸, R⁹, R¹⁰, and R¹¹ can each independently be, at each occurrence, selected from hydrogen, unsubstituted C₁-C₃₄-alkyl, and unsubstituted C₂-C₃₄-alkenyl. Further, R⁶, R⁷, R⁸, R⁹, R¹⁰, and R¹¹ can each independently be, at each occurrence, selected from hydrogen, unsubstituted C₁-C₁₀-alkyl, and unsubstituted C₂-C₁₀-alkenyl. Further, R⁶, R⁷, R⁸, R⁹, R¹⁰, and R¹¹ can each independently be, at each occurrence, selected from hydrogen, and a radical derived from a fatty acid.

R¹² and R₁₃ can each independently be, at each occurrence, selected from hydrogen, C₁-C₁₀-alkyl, —COR¹⁴, —CO₂R¹⁵, —C₁-C₁₀-alkyl-COR¹⁶, and —C₁-C₁₀-alkyl-CO₂R¹⁷. Further, R¹² and R₁₃ can each independently be, at each occurrence, selected from hydrogen, unsubstituted C₁-C₁₀-alkyl, —COR¹⁴, —CO₂R¹⁵, —C₁-C₁₀-alkyl-COR¹⁶, and —C₁-C₁₀-alkyl-CO₂R¹⁷.

R¹⁴, R¹⁵, R¹⁶, and R¹⁷ can each independently be, at each occurrence, selected from hydrogen, unsubstituted alkyl, and unsubstituted alkenyl. Further, R¹⁴, R¹⁵, R¹⁶, and R¹⁷ can each independently be, at each occurrence, selected from hydrogen, unsubstituted C₁-C₃₄-alkyl, and unsubstituted C₂-C₃₄-alkenyl. Additionally, R¹⁴, R¹⁵, R¹⁶, and R¹⁷ can each independently be, at each occurrence, selected from hydrogen, unsubstituted C₁-C₁₀-alkyl, and unsubstituted C₂-C₁₀-alkenyl. Further, R¹⁵ and/or R¹⁷ can be absent where the carboxylic acid moiety is deprotonated.

Imidazoline compounds of the present disclosure can have R¹⁴, R¹⁵, R¹⁶, and R¹⁷ each independently be, at each occurrence, selected from hydrogen, and a radical derived from a fatty acid. Further, R¹⁴, R¹⁵, R¹⁶, and R¹⁷ can each independently be, at each occurrence, selected from hydrogen, C₄-C₃₄-alkyl, and C₄-C₃₄-alkenyl. Additionally, R¹⁴, R¹⁵, R¹⁶, and R¹⁷ can each independently be, at each occurrence, selected from hydrogen; —(CH₂)₃CH₃; —(CH₂)₄CH₃; —(CH₂)₅CH₃; —(CH₂)₆CH₃; —(CH₂)₇CH₃; —(CH₂)₈CH₃; —(CH₂)₉CH₃; —(CH₂)₁₀CH₃; —(CH₂)₁₁CH₃; —(CH₂)₁₂CH₃; —(CH₂)₁₃CH₃; —(CH₂)₁₄CH₃; —(CH₂)₁₅CH₃; —(CH₂)₁₆CH₃; —(CH₂)₁₇CH₃; —(CH₂)₁₈CH₃; —(CH₂)₁₉CH₃; —(CH₂)₂₀CH₃; —(CH₂)₂₁CH₃; —(CH₂)₂₂CH₃; —(CH₂)₂₃CH₃; —(CH₂)₂₄CH₃; —(CH₂)₂₅CH₃; —(CH₂)₂₆CH₃; —(CH₂)₂₇CH₃; —(CH₂)₂₈CH₃; —(CH₂)₂₉CH₃; —(CH₂)₃₀CH₃; —(CH₂)₃₁CH₃; —(CH₂)₃₂CH₃; —(CH₂)₃₃CH₃; —(CH₂)₃₄CH₃; —(CH₂)₂CH═CHCH₂CH═CHCH₂CH═CHCH₂CH═CHCH₂CH═CH(CH₂)₄CH₃; —(CH₂)₂CH═CHCH₂CH═CHCH₂CH—CHCH₂CH—CHCH₂CH═CHCH₂CH—CHCH₂CH₃; —(CH₂)₃CH═CHCH₂CH—CHCH₂CH═CH(CH₂)₇CH₃; —(CH₂)₃CH═CHCH₂CH₂CH═CHCH₂CH═CH(CH₂)₄CH₃; —(CH₂)₃CH═CH(CH₂)₄CH═CHCH₂CH═CH(CH₂)₄CH₃; —(CH₂)₃CH═CHCH₂CH—CHCH₂CH—CHCH₂CH═CH(CH₂)₄CH₃; —(CH₂)₃CH═CHCH₂CH═CHCH₂CH═CHCH₂CH═CHCH₂CH═CHCH₂CH₃; —(CH₂)₃CH═CHCH═CHCH═CHCH═CHCH—CH(CH₂)₄CH₃; —(CH₂)₄CH═CH(CH₂)₈CH₃; —(CH₂)₄CH═CHCH₂CH═CHCH₂CH═CH(CH₂)₄CH₃; —(CH₂)₄CH═CHCH₂CH═CHCH₂CH═CHCH₂CH═CHCH₂CH₃; —(CH₂)₄CH═CHCH₂CH═CHCH₂CH═CHCH₂CH═CHCH₂CH═CHCH₂CH₃; —(CH₂)₄CH═CHCH₂CH—CHCH₂CH═CHCH₂CH═CHCH₂CH═CH(CH₂)₄CH₃; —(CH₂)₄CH—CHCH₂CH═CHCH₂CH═CHCH₂CH═CHCH₂CH═CHCH₂CH═CHCH₂CH₃; —(CH₂)₅CH—CHCH₂CH═CHCH₂CH═CHCH₂CH₃; —(CH₂)₅CH═CHCH₂CH═CHCH₂CH—CHCH₂CH═CH(CH₂)₄CH₃; —(CH₂)₅CH═CHCH₂CH—CHCH₂CH═CHCH₂CH═CHCH₂CH═CHCH₂CH₃; —(CH₂)₆CH═CHCH═CHCH═CH(CH₂)₄CH₃; —(CH₂)₆CH═CHCH₂CH—CHCH₂CH—CHCH₂CH═CH(CH₂)₄CH₃; —(CH₂)₇CH═CH(CH₂)₃CH₃; —(CH₂)₇CH═CH(CH₂)₅CH₃; —(CH₂)₇CH═CH(CH₂)₇CH₃; —(CH₂)₇CH═CHCH═CHCH═CH(CH₂)₃CH₃; —(CH₂)₇CH—CHCH═CH(CH₂)₅CH₃; —(CH₂)₇CH═CHCH₂CH—CH(CH₂)₄CH₃; —(CH₂)₇CH═CHCH₂CH═CH(CH₂)₄CH₃; —(CH₂)₇CH═CHCH—CHCH₂CH₂CH═CHCH₂CH₃; —(CH₂)₇CH═CHCH═CHCH═CHCH═CHCH₂CH₃; —(CH₂)₇CH═CHCH₂CH═CHCH₂CH═CHCH₂CH═CH(CH₂)₄CH₃; —(CH₂)₇CH═CHCH₂CH═CHCH₂CH═CHCH₂CH₃; —(CH₂)₇CH—CHCH₂CH—CHCH₂CH═CHCH₂CH═CHCH₂CH—CHCH₂CH₃; —(CH₂)₉CH═CH(CH₂)₅CH₃; —(CH₂)₉CH═CHCH₂CH═CH(CH₂)₄CH₃; —(CH₂)₉CH═CHCH₂CH═CHCH₂CH═CHCH₂CH₃; —(CH₂)₉CH═CH(CH₂)₇CH₃; —(CH₂)₁₁CH═CH(CH₂)₅CH₃; —(CH₂)₁₁CH═CH(CH₂)₇CH₃; —(CH₂)₁₁CH═CHCH₂CH═CH(CH₂)₄CH₃; and —(CH₂)₁₃CH═CH(CH₂)₇CH₃.

For the imidazolines of formulae (I), (II), and (III), R¹⁴, R¹⁵, R¹⁶, and R¹⁷ can each independently be, at each occurrence, selected from hydrogen, a radical derived from a saturated fatty acid, and a radical derived from an unsaturated fatty acid. Suitable saturated fatty acids include, but are not limited to, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, and hexatriacontylic acid. Suitable unsaturated fatty acids include, but are not limited to, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, hexadecatrienoic acid, stearidonic acid, eicosenic acid, canola acid, eicosatrienoic acid, eicosatetraenoic acid, heneicosapentaenoic acid, clupanodonic acid, osbond acid, (9Z,12Z,15Z,18Z,21Z)-tetracosa-9,12,15,18,21-pentaenoic acid, nisinic acid, γ-linolenic acid, eicosadienoic acid, dihomo-γ-linolenic acid, docosadienoic acid, adrenic acid, tetracosatetraenoic acid, (6Z,9Z,12Z,15Z,18Z)-tetracosa-6,9,12,15,18-pentaenoic acid, (Z)-Eicos-11-enoic acid, mead acid, erucic acid, nervonic acid, rumenic acid, α-calendic acid, β-calendic acid, jacaric acid, α-eleostearic acid, β-eleostearic acid, catalpic acid, punicic acid, rumelenic acid, α-parinaric acid, β-parinaric acid, bosseopentaenoic acid, pinolenic acid, and podocarpic acid.

Further, R¹⁴, R¹⁵, R¹⁶, and R¹⁷ are each independently, at each occurrence, hydrogen or a radical derived from coconut oil, beef tallow, or TOFA.

In some embodiments, the imidazoline is a compound of formula (I), wherein R¹ is unsubstituted C₂-C₆-alkyl; R² is —C₁₇H₃₅, —C₁₇H₃₃, or —C₁₇H₃₁; R³ is —CH₂CH₂CO₂R^{e}, wherein R^{e} is hydrogen (—H), C₁-C₆-alkyl, or R^{e} is absent (e.g., R³ is —CH₂CH₂CO₂⁻); R⁴ is hydrogen; and R⁵ is hydrogen.

In some embodiments, the imidazoline is a compound of formula (I), wherein R¹ is linear C₂-alkyl, substituted with one substituent that is a terminal-N(R¹²)(R¹³), wherein R¹² is hydrogen and R¹³ is —COR¹⁴ wherein R¹⁴ is —C₁₇H₃₅, —C₁₇H₃₃, or —C₁₇H₃₁; R² is —C₁₇H₃₅, —C₁₇H₃₃, or —C₁₇H₃₁; R³ is —CH₂CH₂CO₂R^{e}, wherein R^{e} is hydrogen (—H), C₁-C₆-alkyl, or R^{e} is absent (e.g., R³ is —CH₂CH₂CO₂⁻); R⁴ is hydrogen; and R⁵ is hydrogen.

In certain embodiments, the imidazoline is a compound of formula (I), wherein R¹ is linear C₂-alkyl, substituted with one substituent that is a terminal-N(R¹²)(R¹³), wherein R¹² and R₁₃ are each α-C₂-alkyl-CO₂R¹⁷, wherein R¹⁷ is hydrogen or is absent (e.g., R¹² is —C₂-alkyl-CO₂⁻); R² is —C₁₇H₃₅, —C₁₇H₃₃, or —C₁₇H₃₁; R³ is —CH₂CH₂CO₂R^{e}, wherein R^{e} is hydrogen (—H), C₁-C₆-alkyl, or R^{e} is absent (e.g., R³ is —CH₂CH₂CO₂⁻); R⁴ is hydrogen; and R⁵ is hydrogen.

In some embodiments, the imidazoline is a compound of formula (II), wherein R¹ is unsubstituted C₂-C₆-alkyl; R² is —C₁₇H₃₅, —C₁₇H₃₃, or —C₁₇H₃₁; R³ is —CH₂CH₂CO₂R^{e}, wherein R^{e} is hydrogen (—H), C₁-C₆-alkyl, or R^{e} is absent (e.g., R³ is —CH₂CH₂CO₂⁻); R^{x} is —CH₂CH₂CO₂R^{e}, wherein R^{e} is hydrogen (—H), C₁-C₆-alkyl, or R^{e} is absent (e.g., R^{x} is —CH₂CH₂CO₂⁻); R⁴ is hydrogen; and R⁵ is hydrogen.

In some embodiments, the imidazoline is a compound of formula (II), wherein R¹ is linear C₂-alkyl, substituted with one substituent that is a terminal-N(R¹²)(R¹³), wherein R¹² is hydrogen and R¹³ is —COR¹⁴, wherein R¹⁴ is —C₁₇H₃₅, —C₁₇H₃₃, or —C₁₇H₃₁; R² is —C₁₇H₃₅, —C₁₇H₃₃, or —C₁₇H₃₁; R³ is —CH₂CH₂CO₂R^{e}, wherein R^{e} is hydrogen (—H), C₁-C₆-alkyl, or R^{e} is absent (e.g., R³ is —CH₂CH₂CO₂⁻); R^{x} is —CH₂CH₂CO₂R^{e}, wherein R^{e} is hydrogen (—H), C₁-C₆-alkyl, or R^{e} is absent (e.g., R^{x} is —CH₂CH₂CO₂⁻); R⁴ is hydrogen; and R⁵ is hydrogen.

In certain embodiments, the imidazoline can be a compound of formula (II), wherein R¹ is linear C₂-alkyl, substituted with one substituent that is a terminal —N(R¹²)(R¹³), wherein R¹² and R₁₃ are each α-C₂-alkyl-CO₂R¹⁷, wherein R¹⁷ is hydrogen or is absent (e.g., R¹² is —C₂-alkyl-CO₂″); R² is —C₁₇H₃₅, —C₁₇H₃₃, or —C₁₇H₃₁; R³ is —CH₂CH₂CO₂R^{e}, wherein R^{e} is hydrogen (—H), C₁-C₆-alkyl, or R^{e} is absent (e.g., R³ is —CH₂CH₂CO₂⁻); R^{x} is —CH₂CH₂CO₂R^{e}, wherein R^{e} is hydrogen (—H), C₁-C₆-alkyl, or R^{e} is absent (e.g., R^{x} is —CH₂CH₂CO₂⁻); R⁴ is hydrogen; and R⁵ is hydrogen.

In some embodiments, the imidazoline can be a compound of formula (III), wherein R¹ is unsubstituted C₂-C₆-alkyl; R² is —C₁₇H₃₅, —C₁₇H₃₃, or —C₁₇H₃₁; R⁴ is hydrogen; and R⁵ is hydrogen.

In some embodiments, the imidazoline can be a compound of formula (III), wherein R¹ is linear C₂-alkyl, substituted with one substituent that is a terminal —N(R¹²)(R¹³), wherein R¹² is hydrogen and R¹³ is —COR¹⁴, wherein R¹⁴ is —C₁₇H₃₅, —C₁₇H₃₃, or —C₁₇H₃₁; R² is —C₁₇H₃₅, —C₁₇H₃₃, or —C₁₇H₃₁; R⁴ is hydrogen; and R⁵ is hydrogen.

In certain embodiments, the imidazoline can be a compound of formula (III), wherein R¹ is linear C₂-alkyl, substituted with one substituent that is a terminal —N(R¹²)(R¹³), wherein R¹² and R₁₃ are each α-C₂-alkyl-CO₂R¹⁷, wherein R¹⁷ is hydrogen or is absent (e.g., R¹² is —C₂-alkyl-CO₂⁻); R² is —C₁₇H₃₅, —C₁₇H₃₃, or —C₁₇H₃₁; R⁴ is hydrogen; and R⁵ is hydrogen.

It is to be understood, whether explicitly set forth or not, that formula (I), formula (II), and formula (III) are each intended to encompass the tautomeric, racemic, enantiomeric, diastereomeric, zwitterionic, and salt forms of said formulas. The imidazolines can exist in a zwitterionic form where R³ and/or R^{x} is derived from an acrylic acid.

In accordance with the present disclosure, the corrosion inhibitor compound may be a quaternary amine. Suitable quaternary amines include, but are not limited to, alkyl, hydroxyalkyl, alkylaryl, arylalkyl or arylamine quaternary salts.

Suitable alkyl, hydroxyalkyl, alkylaryl arylalkyl or arylamine quaternary salts include those alkylaryl, arylalkyl and arylamine quaternary salts of the formula [N⁺R^{5a}R^{6a}R^{7a}R^{8a}][X⁻] wherein R^{5a}, R^{6a}, R^{7a}, and R^{8a} contain one to 18 carbon atoms, and X is Cl, Br or I. For the quaternary amine, R^{5a}, R^{6a}, R^{7a}, and R^{8a} can each independently be selected from the group consisting of alkyl (e.g., C₁-C₁₈ alkyl), hydroxyalkyl (e.g., C₁-C₁₈ hydroxyalkyl), and arylalkyl (e.g., benzyl). The mono or polycyclic aromatic amine salt with an alkyl or alkylaryl halide include salts of the formula [N⁺R^{5a}R^{6a}R^{7a}R^{8a}][X⁻] wherein R^{5a}, R^{6a}, R^{7a}, and R^{8a} contain one to 18 carbon atoms, and X is Cl, Br or I.

Suitable quaternary ammonium salts include, but are not limited to, tetramethyl ammonium chloride, tetraethyl ammonium chloride, tetrapropyl ammonium chloride, tetrabutyl ammonium chloride, tetrahexyl ammonium chloride, tetraoctyl ammonium chloride, benzyltrimethyl ammonium chloride, benzyltriethyl ammonium chloride, phenyltrimethyl ammonium chloride, phenyltriethyl ammonium chloride, cetyl benzyldimethyl ammonium chloride, hexadecyl trimethyl ammonium chloride, dimethyl alkyl benzyl quaternary ammonium compounds, monomethyl dialkyl benzyl quaternary ammonium compounds, trimethyl benzyl quaternary ammonium compounds, and trialkyl benzyl quaternary ammonium compounds, wherein the alkyl group can contain between about 1 and about 24 carbon atoms, about 10 and about 18 carbon atoms, or about 12 to about 16 carbon atoms, such as for example, C₁₂₋₁₆ benzyl dimethyl ammonium chloride. Suitable quaternary ammonium compounds (quats) include, but are not limited to, trialkyl, dialkyl, dialkoxy alkyl, monoalkoxy, benzyl, and imidazolinium quaternary ammonium compounds, salts thereof, the like, and combinations thereof. The quaternary ammonium salt can be an alkylamine benzyl quaternary ammonium salt, a benzyl triethanolamine quaternary ammonium salt, or a benzyl dimethylaminoethanolamine quaternary ammonium salt.

The quaternary amine can be a benzalkonium salt represented by the formula:

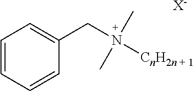

wherein n is 8, 10, 12, 14, 16, or 18; and X is Cl, Br or I.

The quaternary amine can be a mixture of benzalkonium salts wherein n is 8, 10, 12, 14, 16, and 18.

The quaternary amine can be a mixture of benzalkonium salts wherein n is 12, 14, 16, and 18.

The quaternary amine can be a mixture of benzalkonium salts wherein n is 12, 14, and 16.

The quaternary amine can be a mixture of benzalkonium salts wherein n is 12, 14, 16, and 18 and X is Cl.

The quaternary amine can be a mixture of benzalkonium salts wherein n is 12, 14, and 16, and X is Cl.

The quaternary amine can be an alkyl pyridinium quaternary salt such as those represented by the general formula:

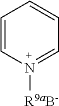

wherein R^{9a} is an alkyl group, an aryl group, or an arylalkyl group, wherein said alkyl groups have from 1 to about 18 carbon atoms and B is Cl, Br or I.

Among these compounds are alkyl pyridinium salts and alkyl pyridinium benzyl quats. Examples include methyl pyridinium chloride, ethyl pyridinium chloride, propyl pyridinium chloride, butyl pyridinium chloride, octyl pyridinium chloride, decyl pyridinium chloride, lauryl pyridinium chloride, cetyl pyridinium chloride, benzyl pyridinium and an alkyl benzyl pyridinium chloride. In some embodiments, the alkyl is a C₁-C₆ hydrocarbyl group.

The compositions disclosed herein may, in some embodiments, include a phosphonium compound, such as a phosphonium salt. Suitable phosphonium salts include, but are not limited to, alkyltris(hydroxyorgano)phosphonium salts, alkenyltris(hydroxyorgano)phosphonium salts, and tetrakis(hydroxyorgano)phosphonium salts. The alkyltris(hydroxyorgano)phosphonium salts can be C₁-C₃-alkyltris(hydroxymethyl)phosphonium salts. The alkenyltris(hydroxyorgano)phosphonium salts can be C₂-C₃-alkenyltris(hydroxymethyl)phosphonium salts. The tetrakis(hydroxyorgano)phosphonium salts can be tetrakis(hydroxymethyl)phosphonium salts, including, but not limited to, tetrakis(hydroxymethyl)phosphonium sulphate (THPS), tetrakis(hydroxymethyl)phosphonium chloride, tetrakis(hydroxymethyl)phosphonium phosphate, tetrakis(hydroxymethyl)phosphonium formate, tetrakis(hydroxymethyl)phosphonium acetate, and tetrakis(hydroxymethyl)phosphonium oxalate. In some embodiments, the phosphonium salt is THPS.

The compound comprising multiple positive charges may be derived from a polyamine through its reactions with an activated olefin and an epoxide, wherein the activated olefin has the following formula:

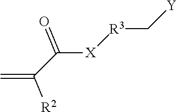

wherein X is NH or O; R² is H, CH₃, or an unsubstituted, linear or branched C₂-C₁₀ alkyl, alkenyl, or alkynyl group; R³ is absent or an unsubstituted, linear C₁-C₃₀ alkylene group; Y is —NR₄R₅R₆⁽⁺⁾; R⁴, R⁵, and R⁶ are independently a C₁-C₁₀ alkyl group; wherein the epoxide has the following formula;

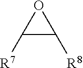

R⁷ is H or alkyl; and R⁸ is alkyl, or —(CH₂)ₖ—O-alkyl, wherein k is an integer of 1-30; wherein the polyamine and activated olefin undergo aza Michael Addition reaction and the polyamine and epoxide undergo ring opening reaction. In some embodiments, the compound comprises a nonionic group.

In some embodiments, the compound has one of the generic formula of NA₂-[R^{10′}]ₙ-NA₂, (RNA)ₙ-RNA₂, NA₂-(RNA)ₙ-RNA₂, or NA₂-(RN(R′))ₙ—RNA₂, wherein R^{10′} is a linear or branched, unsubstituted or substituted C₂-C₁₀ alkylene group, or combination thereof; R is —CH₂—, —CH₂CH₂—, —CH₂CH₂CH₂—, —CH(CH₃) CH₂—, a linear or branched, unsubstituted or substituted C₄-C₁₀ alkylene group, or combination thereof; R′ is —CH₂—, —CH₂CH₂—, —CH₂CH₂CH₂—, —CH(CH₃) CH₂—, a linear or branched, unsubstituted or substituted C₄-C₁₀ alkyl group, RNAB, RNARNAB, or RN(RNAB)₂; n can be from 2 to 1,000,000; A is a combination of H,

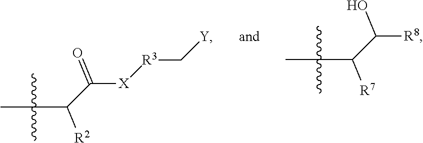

wherein X is NH or O; R² is H, CH₃, or an unsubstituted, linear or branched C₂-C₁₀ alkyl, alkenyl, or alkynyl group; R³ is absent or an unsubstituted, linear C₁-C₃₀ alkylene group; Y is —NR₄R₅R₆⁽⁺⁾; R⁴, R⁵, and R⁶ are independently a C₁-C₁₀ alkyl group; R⁷ is H or alkyl; and R⁸ is alkyl, or —(CH₂) k-O-alkyl, wherein k is an integer of 1-30.

The compound may be a multiple charged cationic compound having a

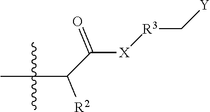

group and a

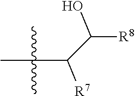

group.

In some aspects, the treatment chemical can be 2-mercaptoethanol, a DETA:TOFA imidazoline, a reaction product of trimethylamine (TEA) and TOFA, a reaction product of TOFA and TEPA, an alkyl pyridine, an ethoxylated branched nonylphenol phosphate ester, a benzyl-(C₁₂ to C₁₈ linear alkyl)-dimethylammonium chloride, 5-carboxy-4-hexyl-2-cyclohexene octanoic acid, 6-carboxy-4-hexyl-2-cyclohexene octanoic acid, maleated TOFA, an acrylated DETA:TOFA imidazoline, and any combination thereof.

In some embodiments, the corrosion inhibitor may be selected from, for example, benzyl ammonium chloride, acrylated imidazoline, 2-mercaptoethanol, a quaternary ammonium compound, a phosphate ester, a substituted aromatic amine, an alkyl pyridine, a fatty acid amine condensate, and any combination thereof.

The compositions disclosed herein may also, in certain embodiments, include an iron chelating agent. For example, the iron chelating agent may include nitrilotriacetic acid, tannic acid, ethylenediaminetetraacetic acid (EDTA), diethylenetriaminepentaacetic acid, TETA hexaacetic acid, 1-hydroxyethylidene-1,1-diphosphonic acid (HEDP), or any combination thereof.

In some embodiments, the iron chelating agent may comprise an EDTA derivative, such as disodium EDTA, calcium disodium EDTA, a tetraammonium salt of EDTA, or any combination thereof.

The presently disclosed corrosion inhibitor compositions are useful for inhibiting corrosion of metal surfaces in contact with any type of corrodent in the medium, such as a metal cation, a metal complex, a metal chelate, an organometallic complex, an aluminum ion, an ammonium ion, a barium ion, a chromium ion, a cobalt ion, a cuprous ion, a cupric ion, a calcium ion, a ferrous ion, a ferric ion, a hydrogen ion, a magnesium ion, a manganese ion, a molybdenum ion, a nickel ion, a potassium ion, a sodium ion, a strontium ion, a titanium ion, a uranium ion, a vanadium ion, a zinc ion, a bromide ion, a carbonate ion, a chlorate ion, a chloride ion, a chlorite ion, a dithionate ion, a fluoride ion, a hypochlorite ion, an iodide ion, a nitrate ion, a nitrite ion, an oxide ion, a perchlorate ion, a peroxide ion, a phosphate ion, a phosphite ion, a sulfate ion, a sulfide ion, a sulfite ion, a hydrogen carbonate ion, a hydrogen phosphate ion, a hydrogen phosphite ion, a hydrogen sulfate ion, a hydrogen sulfite ion, an acid, such as carbonic acid, hydrochloric acid, nitric acid, sulfuric acid, nitrous acid, sulfurous acid, a peroxy acid, or phosphoric acid, ammonia, bromine, carbon dioxide, chlorine, chlorine dioxide, fluorine, hydrogen chloride, hydrogen sulfide, iodine, nitrogen dioxide, nitrogen monoxide, oxygen, ozone, sulfur dioxide, hydrogen peroxide, a polysaccharide, a metal oxide, sand, a clay, silicon dioxide, titanium dioxide, mud, an insoluble inorganic and/or organic particulate, an oxidizing agent, a chelating agent, an alcohol, and any combination of the foregoing.

In some embodiments, the medium can include a corrodent selected from the group consisting of hydrogen sulfide, carbon dioxide, oxygen, sodium chloride, calcium chloride, sulfur dioxide, elemental sulfur, organic acids, and any combination thereof.

The presently disclosed corrosion inhibitor compositions are useful for inhibiting corrosion of surfaces comprising any metal or combination of metals. In some aspects, the metal surface comprises steel, such as stainless steel or carbon steel. In some aspects, the metal surface comprises iron, aluminum, zinc, chromium, manganese, nickel, tungsten, molybdenum, titanium, vanadium, cobalt, niobium, copper, or any combination thereof. The metal surface may also comprise boron, phosphorus, sulfur, silicon, oxygen, nitrogen, and any combination thereof. In some aspects, a pipe, such as a pipeline, a flowline, a downhole tubular, a casing, a tank, or a separator, or any component in fluid communication with the pipe comprises the metal surface.

The compositions of the present disclosure may comprise various amounts of the compounds/components disclosed herein, such as the corrosion inhibitor compound.

For example, a composition may comprise from about 0.5 wt. % to about 100 wt. % of the corrosion inhibitor compound, such as from about 5 wt. % to about 85 wt. %, about 5 wt. % to about 75 wt. %, about 5 wt. % to about 65 wt. %, about 5 wt. % to about 55 wt. %, about 5 wt. % to about 45 wt. %, about 5 wt. % to about 35 wt. %, about 5 wt. % to about 25 wt. %, about 10 wt. % to about 20 wt. %, about 10 wt. % to about 30 wt. %, about 10 wt. % to about 40 wt. %, about 10 wt. % to about 50 wt. %, about 10 wt. % to about 60 wt. %, about 20 wt. % to about 60 wt. %, about 25 wt. % to about 55 wt. %, or about 30 wt. % to about 50 wt. % of the corrosion inhibitor compound.

Furthermore, a composition of the present disclosure may comprise from about 0.5 wt. % to about 50 wt. % of an additional compound(s) and/or component(s), such as from about 0.5 wt. % to about 40 wt. %, about 0.5 wt. % to about 30 wt. %, about 0.5 wt. % to about 20 wt. %, about 0.5 wt. % to about 10 wt. %, about 0.5 wt. % to about 5 wt. %, about 1 wt. % to about 10 wt. %, about 1 wt. % to about 20 wt. %, about 1 wt. % to about 30 wt. %, about 5 wt. % to about 25 wt. %, or about 10 wt. % to about 20 wt. %.

For example, with respect to the iron chelating agent, the composition may include about 5 wt. %, about 7.5 wt. % about 10 wt. %, about 12 wt. %, or about 15 wt. %.

In some embodiments, a composition may comprise from about 10 wt. % to about 50 wt. % of the corrosion inhibitor compound and from about 1 wt. % to about 20 wt. % of the iron chelating agent. In some embodiments, the composition comprises a greater weight percentage of the corrosion inhibitor compound than the iron chelating agent.

In certain embodiments, a composition comprises from about 30 wt. % to about 50 wt. % of the corrosion inhibitor compound and from about 1 wt. % to about 10 wt. % of the iron chelating agent.

When the compositions disclosed herein include the iron chelating agent, the may comprise a ratio of corrosion inhibitor compound to iron chelating agent of about 0.5:1 to about 50:1. In some embodiments, the weight ratio is about 0.5:1 to about 40:1, about 0.5:1 to about 30:1, about 0.5:1 to about 20:1, about 0.5:1 to about 10:1, about 0.5:1 to about 5:1, about 0.5:1 to about 1:1, about 1:1 to about 30:1, about 1:1 to about 20:1, about 1:1 to about 10:1, about 1:1 to about 5:1, about 2:1 to about 5:1, about 2:1 to about 10:1, about 2:1 to about 15:1, or from about 2:1 to about 20:1.

The compositions of the present disclosure may further comprise a solvent. Suitable solvents include, but are not limited to, an alcohol, a hydrocarbon, a ketone, an ether, an aromatic, an amide, a nitrile, a sulfoxide, an ester, a glycol ether, water, and combinations thereof.

For example, the solvent can be water, isopropanol, methanol, ethanol, 2-ethylhexanol, heavy aromatic naphtha, toluene, ethylene glycol, ethylene glycol monobutyl ether (EGMBE), diethylene glycol monoethyl ether, xylene, or any combination thereof.

Representative polar solvents suitable for formulation with the composition include water, brine, seawater, an alcohol (including straight chain or branched aliphatic, such as methanol, ethanol, propanol, isopropanol, butanol, 2-ethylhexanol, hexanol, octanol, decanol, 2-butoxyethanol, etc.), a glycol and a glycol derivative (e.g., ethylene glycol, 1,2-propylene glycol, 1,3-propylene glycol, ethylene glycol monobutyl ether, etc.), a ketone (such as cyclohexanone, diisobutylketone), N-methylpyrrolidinone (NMP), N,N-dimethylformamide, and the like.

Representative non-polar solvents suitable for formulation with the composition include an aliphatic, such as pentane, hexane, cyclohexane, methylcyclohexane, heptane, decane, dodecane, diesel, and the like; and an aromatic, such as toluene, xylene, heavy aromatic naphtha, a fatty acid derivative (e.g., an acid, an ester, an amide), and the like.

In some embodiments, the solvent is methanol, isopropanol, 2-ethylhexanol, or a combination thereof. In certain embodiments, the solvent is methanol, isopropanol, 2-ethylhexanol, water, or a combination thereof.

A composition of the present disclosure may include from about 0 wt. % to about 99 wt. % of the solvent, such as from about 1 wt. % to about 99 wt. % of the solvent, about 5 wt. % to about 95 wt. %, about 5 wt. % to about 85 wt. %, about 5 wt. % to about 75 wt. %, about 5 wt. % to about 65 wt. %, about 5 wt. % to about 55 wt. %, about 5 wt. % to about 45 wt. %, about 5 wt. % to about 35 wt. %, about 5 wt. % to about 25 wt. %, about 5 wt. % to about 15 wt. %, about 15 wt. % to about 95 wt. %, about 25 wt. % to about 95 wt. %, about 35 wt. % to about 95 wt. %, about 40 wt. % to about 85 wt. %, or about 40 wt. % to about 80 wt. % of the solvent.

A composition of the present disclosure may comprise about 5 wt. %, about 10 wt. %, about 15 wt. %, about 20 wt. %, about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, about 80 wt. %, about 85 wt. %, about 90 wt. %, about 95 wt. %, or about 99 wt. % of the solvent.

Any composition disclosed herein may comprise (or exclude) an additional treatment chemical. In some embodiments, the treatment chemical is selected from the group consisting of a hydrate inhibitor, an asphaltene inhibitor, a paraffin inhibitor, a biocide, a scale inhibitor, and any combination thereof.

A hydrate inhibitor may include, for example, a mono-alkyl amide, a di-alkyl amide, an alkyl quaternary ammonium salt, and any combination thereof.

An asphaltene inhibitor may include, for example, an alkylphenol/formaldehyde resin, a polyisobutylene esters, a polyisobutylene imides, a polyalkyl acrylate, and any combination thereof.

A paraffin inhibitor may include, for example, a polyalkyl acrylate, an olefin/maleic anhydride polymer, and any combination thereof.

A biocide may include, for example, glutaraldehyde, tetrakis(hydroxymethyl)phosphonium sulphate, a quaternary ammonium compound, and any combination thereof.

A scale inhibitor may include, for example, a phosphonate, a sulfonate, a phosphate, a phosphate ester, a polymer comprising a phosphonate or phosphonate ester group, a polymeric organic acid, a peroxycarboxylic acid, and any combination thereof. In some embodiments, the scale inhibitor may be selected from a compound comprising an amine and/or a quaternary amine, nitrilotriacetic acid (NTA), ethylenediaminetetraacetic acid (EDTA), DETA phosphonate, and any combination thereof.

In some embodiments, the scale inhibitor is an acid-based scale inhibitor, such as phosphonic acid. In some embodiments, the scale inhibitor comprises an anionic group. The anionic group may comprise, for example, a carboxylate group or a sulfate group. In some embodiments, the scale inhibitor may include a phosphorous atom, a phosphorous-oxygen double bond, and/or a phosphono group.

In some embodiments, the scale inhibitor is selected from the group consisting of hexamethylene diamine tetrakis(methylene phosphonic acid), diethylene triamine tetra (methylene phosphonic acid), diethylene triamine penta (methylene phosphonic acid), polyacrylic acid (PAA), phosphino carboxylic acid (PPCA), diglycol amine phosphonate (DGA phosphonate), 1-hydroxyethylidene 1,1-diphosphonate (HEDP phosphonate), bisaminoethylether phosphonate (BAEE phosphonate), 2-acrylamido-2-methyl-1-propanesulphonic acid (AMPS), and any combination thereof.

In certain embodiments, the scale inhibitor is a polymer comprising an anionic monomer. The anionic monomer may be selected from, for example, acrylic acid, methacrylic acid, vinyl sulfonic acid, vinyl phosphonic acid, maleic anhydride, itaconic acid, crotonic acid, maleic acid, fumaric acid, styrene sulfonic acid, and any combination thereof.

In some embodiments, a composition disclosed herein may comprise (or exclude) an additional treatment chemical selected from a fouling control agent, a corrosion inhibitor intensifier, a biocide, a preservative, an acid, a hydrogen sulfide scavenger, a surfactant, an asphaltene inhibitor, a paraffin inhibitor, a scale inhibitor, a gas hydrate inhibitor, a pH modifier, an emulsion breaker, a reverse emulsion breaker, a coagulant/flocculant agent, an emulsifier, a water clarifier, a dispersant, an antioxidant, a polymer degradation prevention agent, a permeability modifier, a foaming agent, an antifoaming agent, a CO₂ scavenger, an O₂ scavenger, a gelling agent, a lubricant, a friction reducing agent, a salt, a clay stabilizer, a bactericide, a salt substitute, a relative permeability modifier, a breaker, a fluid loss control additive, an iron control agent, a drag reducing agent, a flow improver, a viscosity reducer, a solvent, and any combination thereof.

In some embodiments, the compositions of the present disclosure may be in the form of a liquid, a gel, or a mixture thereof.

In certain embodiments, the compositions disclosed herein comprise a pH from about 1 to about 11. In some embodiments, the pH of the composition may be from about 1 to about 10, from about 1 to about 9, from about 1 to about 8, from about 1 to about 7, from about 1 to about 6, from about 1 to about 5, from about 2 to about 10, from about 2 to about 9, from about 3 to about 10, from about 3 to about 9, or about 3 to about 8.

The present disclosure also provides methods of inhibiting corrosion of a metal surface in contact with a medium. The methods comprise adding an effective amount of a composition to the medium, wherein the composition comprises, consists of, or consists essentially of a corrosion inhibitor compound/reaction product disclosed herein, optionally combined with a solvent and/or any additional compound or component disclosed herein. The composition may be added to the medium continuously, intermittently, automatically, and/or manually.

In some embodiments, the effective amount added to the medium is from about 1 ppm to about 50,000 ppm or about 10 ppm to about 500 ppm. For example, the effective amount may be from about 1 ppm to about 45,000 ppm, from about 1 ppm to about 40,000 ppm, from about 1 ppm to about 35,000 ppm, from about 1 ppm to about 30,000 ppm, from about 1 ppm to about 25,000 ppm, from about 1 ppm to about 20,000 ppm, from about 1 ppm to about 15,000 ppm, from about 1 ppm to about 10,000 ppm, from about 1 ppm to about 5,000 ppm, from about 1 ppm to about 2,000 ppm, from about 1 ppm to about 1,000 ppm, from about 1 ppm to about 500 ppm, from about 100 ppm to about 250 ppm, from about 1 ppm to about 100 ppm, from about 100 ppm to about 50,000 ppm, from about 500 ppm to about 50,000 ppm, from about 1,000 ppm to about 50,000 ppm, from about 5,000 ppm to about 50,000 ppm, from about 10,000 ppm to about 50,000 ppm, or from about 25,000 ppm to about 50,000 ppm.

In some embodiments, the medium is an aqueous medium, such as produced water, seawater, municipal water, “gray” water, brackish water, fresh water, recycled water, salt water, surface water, condensed water, cooling water, injection water, wastewater, geothermal water, sewage water, nuclear cooling water, connate, groundwater, or any combination of the foregoing. The aqueous medium may be a continuously flowing medium, such as produced water flowing from a subterranean reservoir and into or through a pipe or tank. The aqueous medium may also be, for example, wastewater isolated from a continuous manufacturing process flowing into a wastewater treatment apparatus. In other embodiments, the aqueous medium is a batch, or plug, substantially disposed in a batchwise or static state within a metal containment.

The presently disclosed compositions are useful for inhibiting corrosion of metal surfaces in contact with any type of corrodent in the medium, such as metal cations, metal complexes, metal chelates, organometallic complexes, aluminum ions, ammonium ions, barium ions, chromium ions, cobalt ions, cuprous ions, cupric ions, calcium ions, ferrous ions, ferric ions, hydrogen ions, magnesium ions, manganese ions, molybdenum ions, nickel ions, potassium ions, sodium ions, strontium ions, titanium ions, uranium ions, vanadium ions, zinc ions, bromide ions, carbonate ions, chlorate ions, chloride ions, chlorite ions, dithionate ions, fluoride ions, hypochlorite ions, iodide ions, nitrate ions, nitrite ions, oxide ions, perchlorate ions, peroxide ions, phosphate ions, phosphite ions, sulfate ions, sulfide ions, sulfite ions, hydrogen carbonate ions, hydrogen phosphate ions, hydrogen phosphite ions, hydrogen sulfate ions, hydrogen sulfite ions, an acid, such as carbonic acid, hydrochloric acid, nitric acid, sulfuric acid, nitrous acid, sulfurous acid, a peroxy acid, or phosphoric acid, ammonia, bromine, carbon dioxide, chlorine, chlorine dioxide, fluorine, hydrogen chloride, hydrogen sulfide, iodine, nitrogen dioxide, nitrogen monoxide, oxygen, ozone, sulfur dioxide, hydrogen peroxide, polysaccharides, metal oxides, sands, clays, silicon dioxide, titanium dioxide, muds, insoluble inorganic and/or organic particulates, an oxidizing agent, a chelating agent, an alcohol, and any combination of the foregoing.

In some embodiments, the medium is an aqueous medium with a pH of about 1 to about 14. For example, the aqueous medium may have a pH less than about 7 or greater than about 7. In some embodiments, the pH of the aqueous medium is between about 1 and about 6, about 2 and about 6, about 3 and about 6, about 4 and about 6, and about 5 and about 6. In some embodiments, the pH of the aqueous medium is between about 7 and about 14. For example, the pH may be about 7 to about 12, about 7 to about 10, or about 7 to about 8.

In some embodiments, the aqueous medium comprises from about 1 ppm to about 50,000 ppm, by weight or by volume, of the corrosion inhibitor compound/reaction product(s) disclosed herein. In some embodiments, the aqueous medium comprises from about 1 ppm to about 40,000 ppm, from about 1 ppm to about 30,000 ppm, from about 1 ppm to about 20,000 ppm, from about 1 ppm to about 10,000 ppm, from about 1 ppm to about 5,000 ppm, from about 1 ppm to about 1,000 ppm, from about 1 ppm to about 500 ppm, or from about 1 ppm to about 100 ppm of the corrosion inhibitor compound/reaction product(s) disclosed herein.

The presently disclosed compositions are useful for inhibiting corrosion of any metal surfaces. In some embodiments, the metal surface comprises steel, such as stainless steel or carbon steel. In some embodiments, the metal surface comprises iron, aluminum, zinc, chromium, manganese, nickel, tungsten, molybdenum, titanium, vanadium, cobalt, niobium, or copper. The metal surface may also comprise any combination of the foregoing metals and/or any one or more of boron, phosphorus, sulfur, silicon, oxygen, and nitrogen. In some embodiments, a pipe or a tank (e.g., railroad tank car or a tank truck/tanker) comprises the metal surface.

In some embodiments, the methods disclosed herein further comprise adding a component to the medium. The component may be added before, after, and/or with the composition. The component may be added continuously, automatically, intermittently, and/or manually. In some embodiments, the composition comprises the component. In some embodiments, the composition consists of or consists essentially of the corrosion inhibitor compound/reaction product, a solvent, and a component.

Illustrative, non-limiting examples of components include a fouling control agent, an additional corrosion inhibitor, a biocide, a preservative, an acid, a hydrogen sulfide scavenger, a surfactant, an asphaltene inhibitor, a paraffin inhibitor, a scale inhibitor, a gas hydrate inhibitor, a pH modifier, an emulsion breaker, a reverse emulsion breaker, a coagulant/flocculant agent, an emulsifier, a water clarifier, a dispersant, an antioxidant, a polymer degradation prevention agent, a permeability modifier, a foaming agent, an antifoaming agent, a CO₂ scavenger, an O₂ scavenger, a gelling agent, a lubricant, a friction reducing agent, a salt, and any combination thereof.

The additional corrosion inhibitor may comprise, for example, an imidazoline compound, a pyridinium compound, a quaternary ammonium compound, a phosphate ester, an amine, an amide, a carboxylic acid, a thiol, and any combination thereof.

The fouling control agent may comprise, for example, a quaternary compound.

Illustrative, non-limiting examples of biocides include chlorine, hypochlorite, ClO₂, bromine, ozone, hydrogen peroxide, peracetic acid, peroxycarboxylic acid, peroxycarboxylic acid composition, peroxysulphate, glutaraldehyde, dibromonitrilopropionamide, isothiazolone, terbutylazine, polymeric biguanide, methylene bisthiocyanate, tetrakis hydroxymethyl phosphonium sulphate, and any combination thereof.

The acid may comprise, for example, hydrochloric acid, hydrofluoric acid, citric acid, formic acid, acetic acid, or any combination thereof.

The hydrogen sulfide scavenger may comprise, for example, an oxidant, inorganic peroxide, chlorine dioxide, a C₁-C₁₀ aldehyde, formaldehyde, glyoxal, glutaraldehyde, acrolein, methacrolein, a triazine, or any combination thereof.

The surfactant may be non-ionic, cationic, anionic, amphoteric, or zwitterionic.

When the composition comprises a component (or combination of components), it generally comprises from about 0.1 wt. % to about 20 wt. % of the component. For example, the composition may comprise from about 0.1 wt. % to about 15 wt. %, from about 0.1 wt. % to about 10 wt. %, from about 0.1 wt. % to about 5 wt. %, from about 0.1 wt. % to about 1 wt. %, from about 1 wt. % to about 5 wt. %, or from about 1 wt. % to about 10 wt. % of the component.

The composition (and optional component if separate from the composition) may be added to the medium neat, dissolved in a solvent, partially dissolved in a solvent, and/or dispersed in a solvent. The addition may involve manual addition, automatic addition, dripping, pouring, spraying, pumping, injecting, or otherwise adding the composition and optional component to the medium and/or the metal surface. In some embodiments, the composition may be heated, such as from about 30° C. to 100° C., prior to addition. In some embodiments, the composition is added directly to the metal surface instead of or in addition to the medium.

In some embodiments, the medium and/or metal surface to be treated with the presently disclosed composition may be located in a cooling water system, a boiler water system, a petroleum well, a downhole formation, a geothermal well, a mineral washing process, a flotation and benefaction process, a papermaking process, a gas scrubber, an air washer, a continuous casting processes, an air conditioning and refrigeration process, a water reclamation process, a water purification process, a membrane filtration process, a clarifier, a municipal sewage treatment process, a municipal water treatment process, or a potable water system.

The foregoing may be better understood by reference to the following examples, which are intended for illustrative purposes and are not intended to limit the scope of the disclosure or its application in any way.

### EXAMPLES

Corrosion inhibition performance of various compositions of the present disclosure were evaluated. Two methods of testing performance were utilized.
Method 1:

A corrosion bubble cell test was performed using the following conditions to evaluate the corrosion inhibition performance of Composition 1, Composition 2, and Composition 3 on a carbon steel electrode (C1018 grade). The corrosion rate can be obtained electrochemically using linear polarization resistance (LPR) methodology.

Bubble test conditions were as follows: about 66° C., 80% synthetic brine and 20% LVT 200 (hydrocarbon) saturated with CO₂. A pre-corrosion time (i.e., with no corrosion inhibitor) was conducted for about 2 hours before adding about 10 ppm of the experimental blends (Composition 1, Composition 2, Composition 3) being added, which equates to about 2 ppm of the active chemistries with about 0.5 ppm of 2-mercaptoethanol being introduced into the test cell.

A summary of bubble test results is shown in Table 1 below.

TABLE 1

| | 2- | Chemistry | Ave. Blank | Corrosion | % |
|---|---|---|---|---|---|
| | mercaptoethanol | Activity | Corrosion | Rate After | Pro- |
| | (%) | % | Rate | 22 Hr | tection |
| Composition | 5 | 20 | 194 | 1.5 | 99.3 |
| 1 | | | | | |
| Composition | 5 | 20 | 202 | 3.4 | 98.3 |
| 2 | | | | | |
| Composition | 5 | 20 | 196 | 2.2 | 98.9 |
| 3 | | | | | |

Method 2:

Another method of testing the corrosion included Corrosion Buchi autoclave tests, which were performed using the following conditions to evaluate corrosion performance of the compositions on a carbon steel electrode (×65 grade). The corrosion rate can be calculated based on weight loss and the pit depth was obtained using Bruker Npflex Profilometer.

The Buchi autoclave test conditions were as follows: about 121° C., 100% pre-partitioned synthetic brine, about 16 psi CO₂, about 100 psi N₂, about 130 Pa shear stress, about 7 days. After the tests, two X-65 flat coupons/per test were cleaned for mass loss to calculate general corrosion rate and then scanned using the Bruker Npflex Profilometer for pit depth.

The pre-partition conditions were about 60° C., about 40% synthetic brine and about 60% hydrocarbon. About 150 ppm of experimental Composition 1, Composition 2, or Composition 3 were mixed at about 500 rpm for about 30 minutes and allowed to separate prior to transferring the brine to the autoclave.

A summary of the Buchi autoclave test results are presented in Table 2.

TABLE 2

| | Chemistry | | Corrosion Rate | Ave. Pit |
|---|---|---|---|---|
| | Activity | Dosage | After 7 Days | Depth After |
| | % | (ppm) | (mpy) | 7 Days (μm) |
| Blank | 0 | 0 | 4.39 | 44 |
| Composition | 25 | 150 | 1.67 | 10 |
| 1 | | | | |
| Composition | 25 | 150 | 1.91 | 10 |
| 2 | | | | |
| Composition | 25 | 150 | 1.09 | 13 |
| 3 | | | | |

All of the compositions and methods disclosed and claimed herein can be made and executed without undue experimentation in light of the present disclosure. While this invention may be embodied in many different forms, there are described in detail herein specific preferred embodiments of the invention. The present disclosure is an exemplification of the principles of the invention and is not intended to limit the invention to the particular embodiments illustrated. In addition, unless expressly stated to the contrary, use of the term “a” is intended to include “at least one” or “one or more.” For example, “a reaction product” is intended to include “at least one reaction product” or “one or more reaction products.”

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Any composition disclosed herein may comprise, consist of, or consist essentially of any element, component and/or ingredient disclosed herein or any combination of two or more of the elements, components or ingredients disclosed herein.

Any method disclosed herein may comprise, consist of, or consist essentially of any method step disclosed herein or any combination of two or more of the method steps disclosed herein.

The transitional phrase “comprising,” which is synonymous with “including,” “containing,” or “characterized by,” is inclusive or open-ended and does not exclude additional, un-recited elements, components, ingredients and/or method steps.

The transitional phrase “consisting of” excludes any element, component, ingredient, and/or method step not specified in the claim.

The transitional phrase “consisting essentially of” limits the scope of a claim to the specified elements, components, ingredients and/or steps, as well as those that do not materially affect the basic and novel characteristic(s) of the claimed invention.

Unless specified otherwise, all molecular weights referred to herein are weight average molecular weights and all viscosities were measured at 25° C. with neat (not diluted) polymers.

As used herein, the term “about” refers to the cited value being within the errors arising from the standard deviation found in their respective testing measurements, and if those errors cannot be determined, then “about” may refer to, for example, within 5%, 4%, 3%, 2%, or 1% of the cited value.

Furthermore, the invention encompasses any and all possible combinations of some or all of the various embodiments described herein. It should also be understood that various changes and modifications to the presently preferred embodiments described herein will be apparent to those skilled in the art. Such changes and modifications can be made without departing from the spirit and scope of the invention and without diminishing its intended advantages. It is therefore intended that such changes and modifications be covered by the appended claims.

1. A method of inhibiting corrosion of a metal surface in contact with a medium, comprising:
adding an effective amount of a composition to the medium, wherein the composition comprises a maleated fatty imidazoline compound, a maleated fatty imidazoline acrylate compound, a maleated fatty imidazoline acetate compound, or a combination thereof.

2. The method of claim 1, further comprising forming the maleated fatty imidazoline by reacting one or more of maleated tall oil fatty acid, soya acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, hexadecatrienoic acid, stearidonic acid, eicosenic acid, canola acid, eicosatrienoic acid, eicosatetraenoic acid, heneicosapentaenoic acid, clupanodonic acid, osbond acid, (9Z,12Z,15Z,18Z,21Z)-tetracosa-9,12,15,18,21-pentaenoic acid, nisinic acid, γ-linolenic acid, eicosadienoic acid, dihomo-γ-linolenic acid, docosadienoic acid, adrenic acid, tetracosatetraenoic acid, (6Z,9Z,12Z,15Z,18Z)-tetracosa-6,9,12,15,18-pentaenoic acid, (Z)-Eicos-11-enoic acid, mead acid, erucic acid, nervonic acid, rumenic acid, α-calendic acid, β-calendic acid, jacaric acid, α-eleostearic acid, β-eleostearic acid, catalpic acid, punicic acid, rumelenic acid, α-parinaric acid, β-parinaric acid, bosseopentaenoic acid, pinolenic acid, and/or podocarpic acid, or any combinations thereof, with one or more of diethylenetriamine (DETA), triethylenetetramine (TETA), or tetraethylenepentamine (TEPA) to form a reaction product.

3. The method of claim 2, further comprising forming the maleated fatty imidazoline acrylate compound by reacting the maleated tall oil fatty acid with one or more of DETA, TETA, or TEPA to form the reaction product and reacting the reaction product with acrylic acid.

4. The method of claim 2, further comprising forming the maleated fatty imidazoline acetate compound by reacting the maleated tall oil fatty acid with one or more of DETA, TETA, and TEPA to form the reaction product and reacting the reaction product with acetic acid.

5. The method of claim 2, further comprising reacting the maleated soya acid with one or more of the DETA, TETA, and TEPA to form a reaction product.

6. The method of claim 5, wherein the reaction product of maleated soya acid and DETA comprises the following structure:

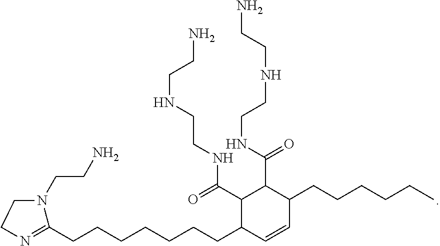 [SMILES: CCCCCCC1C=CC(CCCCCCCC2=NCCN2CCN)C(C(=O)NCCNCCN)C1C(=O)NCCNCCN]

7. The method of claim 5, wherein the reaction product of maleated soya acid and TETA comprises the following structure:

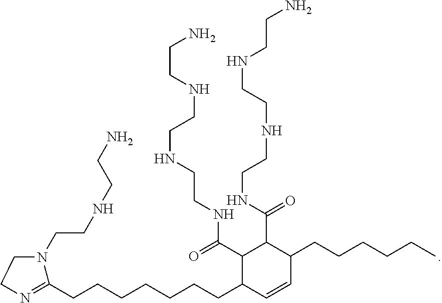 [SMILES: CCCCCCC1C=CC(CCCCCCCC2=NCCN2CCNCCN)C(C(=O)NCCNCCNCCN)C1C(=O)NCCNCCNCCN]

8. The method of claim 5, wherein the reaction product of maleated soya acid and TEPA comprises the following structure:

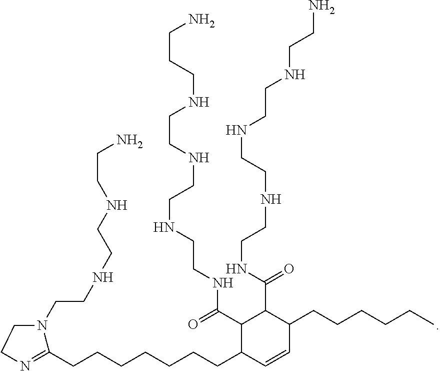 [SMILES: CCCCCCC1C=CC(CCCCCCCC2=NCCN2CCNCCNCCN)C(C(=O)NCCNCCNCCNCCCN)C1C(=O)NCCNCCNCCNCCN]

9. The method of claim 5, further comprising forming the maleated fatty imidazoline acrylate compound by reacting the maleated soya acid with one or more of DETA, TETA, and TEPA to form the reaction product and reacting the reaction product with acrylic acid.

10. The method of claim 5, further comprising forming the maleated fatty imidazoline acetate compound by reacting the maleated soya acid with one or more of DETA, TETA, and TEPA to form the reaction product and reacting the reaction product with acetic acid.

11. The method of claim 1, further comprising forming the maleated fatty imidazoline by reacting one or more of maleated tall oil fatty acid, soya acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, hexadecatrienoic acid, stearidonic acid, eicosenic acid, canola acid, eicosatrienoic acid, eicosatetraenoic acid, heneicosapentaenoic acid, clupanodonic acid, osbond acid, (9Z,12Z,15Z,18Z,21Z)-tetracosa-9,12,15,18,21-pentaenoic acid, nisinic acid, γ-linolenic acid, eicosadienoic acid, dihomo-γ-linolenic acid, docosadienoic acid, adrenic acid, tetracosatetraenoic acid, (6Z,9Z,12Z,15Z,18Z)-tetracosa-6,9,12,15,18-pentaenoic acid, (Z)-Eicos-11-enoic acid, mead acid, erucic acid, nervonic acid, rumenic acid, α-calendic acid, β-calendic acid, jacaric acid, α-eleostearic acid, β-eleostearic acid, catalpic acid, punicic acid, rumelenic acid, α-parinaric acid, β-parinaric acid, bosseopentaenoic acid, pinolenic acid, and/or podocarpic acid, or any combinations thereof, with a mixture of DETA, TETA, or TEPA to form a reaction product.

12. The method of claim 11, further comprising forming the maleated fatty imidazoline acrylate compound by reacting the maleated tall oil fatty acid with the mixture comprising DETA, TETA, and TEPA to form the reaction product and reacting the reaction product with acrylic acid.

13. The method of claim 11, wherein the reaction product comprises the following structure:

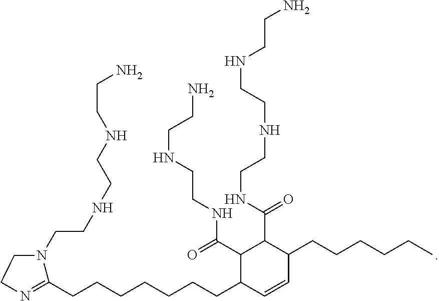 [SMILES: CCCCCCC1C=CC(CCCCCCCC2=NCCN2CCNCCNCCN)C(C(=O)NCCNCCN)C1C(=O)NCCNCCNCCN]

14. The method of claim 11, further comprising forming the maleated fatty imidazoline acetate compound by reacting the maleated tall oil fatty acid with the mixture comprising DETA, TETA, and TEPA to form the reaction product and reacting the reaction product with acetic acid.

15. The method of claim 11, further comprising reacting the maleated soya acid with the mixture comprising DETA, TETA, and TEPA to form the reaction product.

16. The method of claim 15, further comprising forming the maleated fatty imidazoline acrylate compound by reacting the maleated soya acid with the mixture of DETA, TETA, and TEPA to form the reaction product and reacting the reaction product with acrylic acid.

17. The method of claim 15, further comprising forming the maleated fatty imidazoline acetate compound by reacting the maleated soya acid with the mixture of DETA, TETA, and TEPA to form the reaction product and reacting the reaction product with acetic acid.

18. The method of claim 11, wherein the reaction product comprises one of the following structures:

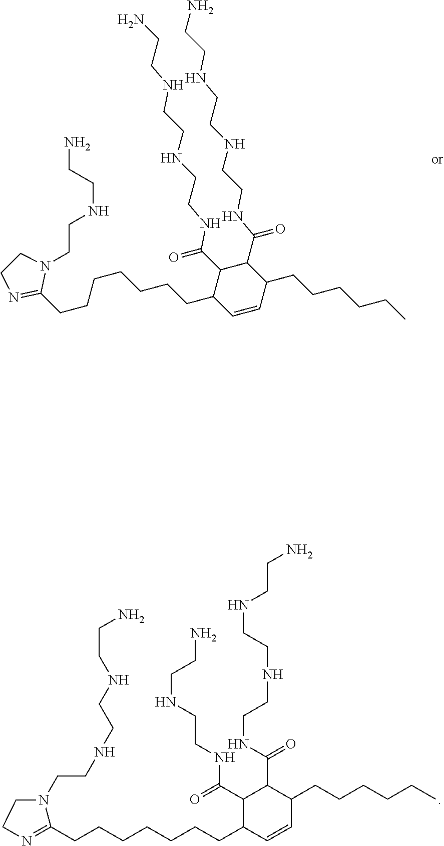 [SMILES: CCCCCCC1C=CC(CCCCCCCC2=NCCN2CCNCCN)C(C(=O)NCCNCCNCCN)C1C(=O)NCCNCCNCCN ; CCCCCCC1C=CC(CCCCCCCC2=NCCN2CCNCCNCCN)C(C(=O)NCCNCCN)C1C(=O)NCCNCCNCCN]

19. The method of claim 1, wherein the maleated fatty imidazoline acrylate compound comprises the following structure:

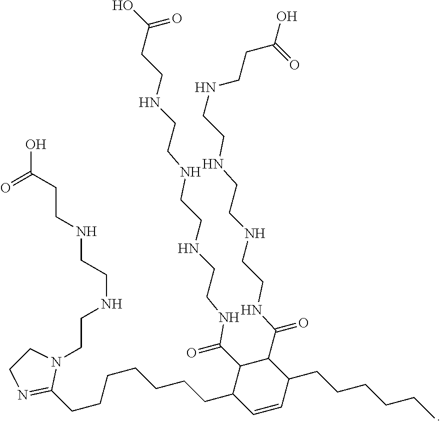 [SMILES: CCCCCCC1C=CC(CCCCCCCC2=NCCN2CCNCCNCCC(=O)O)C(C(=O)NCCNCCNCCNCCC(=O)O)C1C(=O)NCCNCCNCCNCCC(=O)O]

20. The method of claim 1, further comprising adding 2-mercaptoethanol to the medium.
